(19) United States
(12) Patent Application Publication
(10) Pub. No.: US 2008/0070870 A1
(43) Pub. Date: Mar. 20, 2008

(54) Use of omega-3 rich phospholipids in the area of cognitive function

(75) Inventors: Inge Bruheim, Volda (NO); Hogne Hallaraker, Volda (NO); Sebastiano Banno, Cagliari (IT); Mikko Griinari, Espoo (FI)

Correspondence Address: Casimir Jones, S.C., 440 Science Drive, Suite 203, Madison, WI, 53711, US

(73) Assignee: Natural ASA, Lysaker (NO)

(21) Appl. No.: 11/800,371

(22) Filed: May 4, 2007

Related U.S. Application Data
(60) Provisional application No. 60/798,026, filed on May 5, 2006.
(60) Provisional application No. 60/798,027, filed on May 5, 2006.
(60) Provisional application No. 60/798,030, filed on May 5, 2006.
(60) Provisional application No. 60/872,096, filed on Dec. 1, 2006.

(51) Int. Cl. A61K 31/661; A61P 25/00

(52) U.S. Cl. 514/121

(57) ABSTRACT

The invention disclosed describes the of omega-3 rich phospholipids for treating symptoms of cognitive dysfunction in children, children with developmental cognitive disorder, children with autism, elderly subjects with Alzheimer's disease and elderly subjects with age associated cognitive decline.

This application claims the benefit of U.S. Provisional Applications 60/798,026, 60/798,027, and 60/798,030, all filed May 5, 2006, and U.S. Provisional Application 60/872,096, filed Dec. 1, 2006, each of which is incorporated herein by reference in their entirety.

### FIELD OF THE INVENTION

The invention relates to the use of omega-3 fatty acid compositions, and in particular to phospholipid compositions comprising omega-3 fatty acids, to reduce symptoms of cognitive dysfunction in healthy children and children with developmental cognitive disorders as well as methods to reduce symptoms of aged associative cognitive decline and Alzheimer's disease in elderly subjects.

### BACKGROUND OF THE INVENTION

Phospholipids can be isolated from a number of different natural sources such as fish, crustaceans such as Antarctic krill and algae (marine phospholipids). Other sources can be soy, sun flower and maize (vegetable phospholipids). In addition phospholipids can be obtained from eggs. Phospholipids with pre-determined fatty acid residues, so called functional phospholipids, can also be obtained using chemical or enzyme catalyzed processes [1-5]. The uses of phospholipids enriched with omega-3 fatty acids EPA/DHA have been contemplated and explored for several years both for naturally extracted [6-9] and synthetic marine phospholipids [10]. Benefits in the areas of cognition, anti-inflammation and cardiovascular disease have been obtained. The driving force behind these developments has been data indicating that phospholipids are superior carriers of fatty acids into tissue such as red blood cells [11] and brain [12] compared to triacylglycerides (TAG). The data suggest that marine phospholipids are more bioactive than fish oil as they are creating a stronger biological effect with the same dose. These observations in combination with an increasing number of scientific publications documenting positive health effects and the nutritional importance of omega-3 fatty acids [13-14] have fueled the research in the area of omega-3 rich functional phospholipids.

The brain and retina possess the highest concentrations of DHA in humans and animals [15]. In fact, DHA supplementation in gestation and lactation improve visual performance in developing dogs [40]. Previous research using rodent models show that age-associated memory loss may be improved with DHA supplementation [43]. Human research has furthermore demonstrated a correlation between low plasma DHA levels and memory impairment, as well as an association between low levels of LCPUFA consumption and Alzheimer's disease [16]. Previously, the use of marine phospholipids in the area of cognitive function has been investigated. Methods to treat adult psychiatric disorders, neurological disorders and several childhood disorders such as attention deficit hyperactivity disorder (ADHD), dyslexia, dyspraxia and autistic spectrum disorders (ASD) using omega-3 rich phospholipids have been disclosed previously [6,8,10]. In addition, the use of omega-3 rich phospholipids to alleviate inflammation has been disclosed [8]. Actually, omega-3 fatty acids are well-known for their anti-inflammatory properties, as that was one of the earliest identified biological actions of theses fatty acids. Furthermore, it has been shown that omega-3 fatty acids alleviate the symptoms of a series of autoimmune, atherosclerotic and inflammatory diseases [17-19]. Previously, much attention has focused on pro-inflammatory pathways that initiate inflammation, however relatively little is known about the mechanisms that switch off inflammation and resolve the inflammatory response. The transcription factor NF-VB is thought to have a central role in the induction of pro-inflammatory gene expression and has attracted interest as a new target for the treatment of inflammatory disease [20]. Fish oil has anti-inflammatory properties, however new research has shown that it is the fatty acid EPA that is responsible for the prevention of NF-κB activation [21]. Furthermore, recent research has disclosed inflammation is at least a part of the etiologic root of several cognitive disorders. Brain inflammation may be linked to ASD, Alzheimer's disease and age associated cognitive decline/age associated memory decline [22-25]. Marine phospholipids are superior carriers of omega-3 fatty acids into tissue such as red blood cells [11] and the brain [12]. In combination with the anti-inflammatory properties of EPA, marine phospholipids especially with a high EPA:DHA ratio may be potent agents for reducing brain inflammation hence reducing the symptoms of several cognitive disorders such as ASD, Alzheimer's disease, Parkinson's disease, ADHD, dementia and aged associated cognitive decline.

### SUMMARY OF THE INVENTION

An embodiment of the invention is a method to reduce symptoms of cognitive dysfunction in a child with ADHD comprising administering an effective amount of a marine phospholipid composition to a subject, said symptoms are selected from the group consisting of ability to complete task, ability to stay on task, ability to follow instructions, ability to complete assignments, long term memory, short term memory, ability to make a decision, ability to follow through on decision, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information

Another embodiment of the invention is a method to reduce symptoms of cognitive dysfunction in a child with ASD comprising administering an effective amount of a marine phospholipid composition to a subject, said symptoms are selected from the group consisting of ability to complete task, ability to stay on task, ability to follow instructions, ability to complete assignments, long term memory, short term memory, ability to make a decision, ability to follow through on decision, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information.

Another embodiment of the invention is a method to reduce symptoms of cognitive dysfunction in a normal child comprising administering an effective amount of a marine phospholipid composition, wherein said symptoms are selected from the group consisting of ability to complete task, ability to stay on task, ability to follow instructions, ability to complete assignments, long term memory, short term memory, ability to make a decision, ability to follow through on decision, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information.

Yet another embodiment of the invention is a method to reduce symptoms of age associated cognitive decline in a human subject comprising administering marine phospholipids, said symptoms are selected from the group consisting of remembering names, remembering numbers, recalling location of objects, remembering specific facts, inability to concentrate, confusion, hallucinations and delusions, altered sensation or perception, impaired recognition (agnosia), aphasia, altered sleep patterns, motor system impairment, impaired skilled motor function, disorientation, memory deficit, absent or impaired language ability, personality changes, behavioral change, lack of spontaneity and deterioration of musculature and mobility.

Yet another embodiment of the invention is a method to reduce symptoms of age associated cognitive decline in an animal, preferably a dog, comprising administering marine phospholipids, said symptoms are selected from the group consisting of remembering names, remembering numbers, recalling location of objects, remembering specific facts, inability to concentrate, confusion, hallucinations and delusions, altered sensation or perception, impaired recognition (agnosia), aphasia, altered sleep patterns, motor system impairment, impaired skilled motor function, disorientation, memory deficit, absent or impaired language ability, personality changes, behavioral change, lack of spontaneity and deterioration of musculature and mobility.

In yet another embodiment of the invention is a method to reduce the symptoms of Alzheimer's disease in a subject comprising administering marine phospholipids, said symptoms are selected from the group consisting remembering names, remembering numbers, recalling location of objects, remembering specific facts, inability to concentrate, confusion, hallucinations and delusions, altered sensation or perception, impaired recognition (agnosia), aphasia, altered sleep patterns, motor system impairment, impaired skilled motor function, disorientation, memory deficit, absent or impaired language ability, personality changes, behavioral change, lack of spontaneity and deterioration of musculature and mobility. Accordingly, in some embodiments, the present invention provides a composition comprising phospholipids having the following structure: 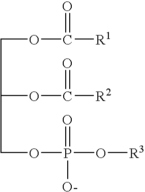
wherein R1 is OH or a fatty acid, R2 is OH or a fatty acid, and R3 is a mixture of H, choline, ethanolamine, inositol and serine, said phospholipid having at least 1% of DHA/EPA at positions R1 and/or R2 and from about 20-50% of OH at positions R1 and/or R2. In some embodiments, the composition further comprises a lipid carrier in a ratio of from 1:10 to 10:1 to said phospholipids. In some embodiments, the lipid carrier and said phospholipids are in a ratio of from about 5:1 to 1:5. In some embodiments, the composition comprises from about 20% to about 90% of said phospholipid composition and from about 10% to about 50% of said lipid carrier. The present invention is not limited to any particular lipid carrier. In some embodiments, the lipid carrier is selected from the group consisting of a triglyceride, a diglyceride, an ethyl ester, and a methyl ester and combinations thereof. In some embodiments, the composition provides higher uptake of omega-3 fatty acids into plasma as compared to natural marine phospholipids when administered to subjects. In some embodiments, the composition improves the AA/EPA ratio in plasma phospholipids when administered to subjects as compared to natural marine phospholipids. In some embodiments, the composition increases the concentration of omega-3 fatty acids in tissues when administered to subjects as compared to natural marine phospholipids. In some embodiments, the composition reduces the concentration of biomarkers of inflammation when administered to subjects as compared to natural marine phospholipids. In some embodiments, the present invention provides a food product comprising the foregoing compositions. In some embodiments, the present invention provides an animal feed comprising the foregoing compositions. In some embodiments, the present invention provides a food supplement comprising the foregoing compositions. In some embodiments, the present invention provides a pharmaceutical composition comprising the foregoing compositions.

In some embodiments, the present invention provides methods of preparing a bioavailable omega-3 fatty acid composition comprising: a) providing a purified phospholipid composition comprising omega-3 fatty acid residues and a purified triglyceride composition comprising omega-3 fatty acid residues; b) combining said phospholipid composition and said triglyceride composition to form a bioavailable omega-3 fatty acid composition. In some embodiments, the bioavailable phospholipid composition is one of the compositions described above. In some embodiments, the methods further comprise the step of encapsulating said bioavailable omega-3 fatty acid composition. In some embodiments, the bioavailable omega-3 fatty acid composition has increased bioavailability as compared to purified triglycerides or phospholipids comprising omega-3 fatty acid residues. In some embodiments, the methods further comprise the step of packaging the bioavailable omega-3 fatty acid composition for use in functional foods. In some embodiments, the methods further comprise the step of assaying the bioavailable omega-3 fatty acid composition for bioavailability. In some embodiments, the methods further comprise administering the bioavailable omega-3 fatty acid composition to a patient. In some embodiments, the present invention provides a food product, animal feed, food supplement or pharmaceutical composition made by the foregoing process.

In some embodiments, the present invention provides methods for reducing symptoms of cognitive dysfunction in a child comprising administering an effective amount of a marine phospholipid composition, wherein said symptoms are selected from the group consisting of ability to complete task, ability to stay on task, ability to follow instructions, ability to complete assignments, psychomotor function, long term memory, short term memory, ability to make a decision, ability to follow through on decision, ability to self-sustain attention, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information. In some embodiments, the child exhibits one or more symptoms of Attention Deficit Hyperactivity Disorder (ADHD), is suspected of having ADHD, or has been diagnosed with ADHD. In some embodiments, the child exhibits one or more symptoms of autistic spectrum disorder, is suspected of having autistic spectrum disorder, or has been diagnosed with autistic spectrum disorder. In further embodiments, the present invention provides methods of increasing cognitive performance in an aging mammal comprising administering an effective amount of a marine phospholipid composition. In some embodiments, the cognitive performance is selected from the group consisting of memory loss, forgetfulness, short-term memory loss, aphasia, disorientation, disinhibition, and behavioral changes. In some embodiments, the mammal is a human. In some embodiments, the mammal is a pet selected from the group consisting of cats and dogs. In some embodiments, the mammal has symptoms of age-associated memory impairment or decline.

The foregoing methods are not limited to the use of any particular marine phospholipid composition. In some embodiments, the marine phospholipid composition comprises phospholipids having the following structure: 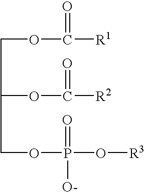
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition further comprises a lipid carrier. In some embodiments, the phospholipid composition is prepared from natural marine phospholipids isolated from a marine organism. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DHA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill.

In some embodiments, the present invention provides methods of treating a subject by administration of a marine phospholipid composition comprising administering a marine phospholipid composition to said subject under conditions such that a desired condition is improved, wherein said conditions is selected from the group consisting of fertility, physical endurance, sports performance, muscle soreness, inflammation, auto-immune stimulation, metabolic syndrome, obesity and type II diabetes. In some embodiments, the subject is a human. In some embodiments, the subject is a companion animal. The present invention is not limited to any particular marine phospholipid composition. In some embodiments, the marine phospholipid composition comprises phospholipids having the following structure: 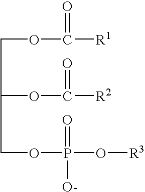
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition is prepared from natural marine phospholipids isolated from a marine organism. In some embodiments, the composition further comprises a lipid carrier. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DHA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill. In some embodiments, the human is a male.

In some embodiments, the present invention provides methods for prophylactically treating a subject by administration of a marine phospholipid composition comprising administering a marine phospholipid composition to a subject under conditions such that an undesirable condition is prevented, wherein said undesirable condition is selected from the group consisting of weight gain, infertility, obesity, metabolic syndrome, diabetes type II, mortality in subjects with a high risk of sudden cardiac death, and induction of sustained ventricular tachycardia. In some embodiments, the subject is at risk for developing a condition selected from the group consisting of weight gain, obesity, metabolic syndrome, diabetes type II, mortality in subjects with a high risk of sudden cardiac death, and induction of sustained ventricular tachycardia.

In some embodiments, the subject is a human. In some embodiments, the subject is a companion animal. The present invention is not limited to any particular marine phospholipid composition. In some embodiments, the marine phospholipid composition comprises phospholipids having the following structure: 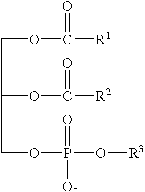
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition is prepared from natural marine phospholipids isolated from a marine organism. In some embodiments, the composition further comprises a lipid carrier. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DHA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill.

### DESCRIPTION OF THE FIGURES

FIG. 1. The composite change score for ADHD subjects receiving placebo and omega-3 phospholipids.

FIG. 2. The composite change score for healthy subjects receiving omega-3.

FIG. 3. Cognitive performance test of aged beagle dogs.

### DEFINITIONS

As used herein, “phospholipid” refers to an organic compound having the following general structure: 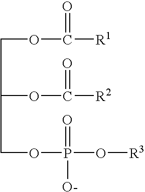
wherein R1 is a fatty acid residue or —OH, 122 is a fatty acid residue or —OH, and R3 is a —H or nitrogen containing compound choline (HOCH₂CH₂N⁺(CH₃)₃OH⁻), ethanolamine (HOCH₂CH₂NH₂), inositol or serine. R1 and 122 cannot simultaneously be —OH. When R3 is an —OH, the compound is a diacylglycerophosphate, while when R3 is a nitrogen-containing compound, the compound is a phosphatide such as lecithin, cephalin, phosphatidyl serine or plasmalogen.

The R1 site is herein referred to as position 1 of the phospholipid, the R2 site is herein referred to as position 2 of the phospholipid, and the R3 site is herein referred to as position 3 of the phospholipid.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term bioavailability refers to the degree and rate at which a substance (as a drug) is absorbed into a living system or is made available at the site of physiological activity.

As used herein, the term “functional food” refers to a food product to which a biologically active supplement has been added.

As used herein, the term “fish oil” refers to any oil obtained from a marine source e.g. tuna oil, seal oil and algae oil.

As used herein, the term “lipase” refers to any enzyme capable of hydrolyzing fatty acid esters

As used herein, the term “food supplement” refers to a food product formulated as a dietary or nutritional supplement to be used as part of a diet.

As used herein, the term “extracted marine phospholipid” refers to a composition characterized by being obtained from a natural source such as krill, fish meal, big brain or eggs.

As used herein, the term “acylation” means fatty acids attached to the phospholipid. 100% acylation means that there are no lyso- or glycero PLs.

As used herein, the term “normal child” means a child that has not been diagnosed as suffering from a cognitive disorder.

As used herein, the term “at risk child” means a child exhibiting one or more symptoms of a cognitive disorder.

As used herein, the term “cognition” is used to describe that operation of the mind process by which we become aware of objects of thought and perception including all aspects of perceiving, thinking and remembering.

As used herein, the term “psychomotor” refers to motor action directly proceeding from mental activity.

### DESCRIPTION OF THE INVENTION

There is increasing evidence that lack of omega-3 fatty acids is associated with childhood developmental cognitive disorders [26-28]. A number of randomized, controlled trials have now addressed this issue reporting that omega-3 supplementation can reduce behavioral and learning difficulties in both ADHD and dyslexia [28-29]. The treatment should preferably consist not only of a mixture of EPA/DHA, but also include omega-6 fatty acids such as 6,9,12 gamma linolenic acid (GLA) [30]. Fontani at al [31] recently reported the results of the only randomized controlled trial of omega-3 and cognition in healthy individuals. Significant improvements on tests of sustained attention and inhibition were observed, as well as reduction in self-rated symptoms of anger, anxiety, fatigue, depression and confusion with a corresponding increase in self-rated levels of vigor for the omega-3 group compared to placebo. However, this manuscript suffered from a number of significant limitations that make interpretation of the results difficult. The study did not include a practice test to minimize “learning effects” prior to the baseline assessment, nor were the results from the placebo group reported or any comparison made between placebo and the active treatment. Previously, in order to investigate the effect of a treatment on a developmental cognitive disorder, behavioral rating scales have been used as the primary outcome measures. Nowadays, many clinical trials of stimulant medication for children with ADHD include cognitive outcome as a co-primary or secondary outcome measure [32]. This is because cognitive dysfunction occurs commonly in children with ADHD, and also there is now substantial evidence that such dysfunction may be responsive to treatment. In addition, cognitive tests may also provide an objective outcome measure and a more direct measurement of the child's brain function than subjective parent or teacher-rated behavioral rating scales.

An embodiment of the invention is to use omega-3 rich phospholipids with EPA/DHA ratio of 2:1 (preferably substantially free of GLA) to reduce the symptoms of cognitive dysfunction in normal children and children with developmental cognitive disorders such as ADHD, dyslexia, dyspraxia and autism. Non-limiting examples of the symptoms that are alleviated are poor long term memory, poor short term memory, inability to make a decision, inability to follow through on a decision, inability to engage in conversations, insensitivity to surroundings and inability to plan a task. This invention discloses that supplementing a child's diet with phospholipid compositions comprising from about 366 mg/d to about 700 mg/d omega-3 per day (EPA:DHA ratio of 2:1) for 12 weeks improves the cognitive function as assessed using conventional rating scales and questionnaires as well as computerized cognitive tests. Previous publications do not disclose the use of omega-3 fatty acids, and in particular phospholipids comprising mega-3 fatty acids, to reduce the symptoms/specific observation criteria that make up the syndrome.

This invention discloses that supplementing a child's diet with phospholipid compositions comprising from about 360 mg to about 700 mg omega-3 per day (EPA:DHA ratio of 2:1) (preferably substantially free of GLA) for 12 weeks results in the improvement in quality of life and quality of health in a child as assessed by the parents using a questionnaire [32]. In the questionnaire, the parent will answer questions related to the child's quality of life, overall health, physical pain, joy over life, ability to concentrate, safety feeling, energy, bodily appearance, ability to gather information, sleep pattern, ability to perform on activities, capacity for school, personal relationships, negative feelings (such as blue mood, despair, anxiety and depression), abdominal discomfort, incidences of constipation, diarrhea, dry mouth, nausea, heartburn, anger, nervousness, binge eating, chest pain, shortness of breath, blurred vision, tremor, memory loss, drowsiness, fatigue, coordination, mental sharpness, hair, skin, nails, eczema and tendency to sweat.

This invention discloses that after administration of marine phospholipids with a EPA/DHA ratio of 2:1 for one week a number of genes involved in the inflammatory response are regulated in a positive way. Furthermore, it is disclosed that marine phospholipids with EPA/DHA ratio of 1:1 do not regulate any genes involved in the inflammatory response. Examples of the proteins regulated by the high EPA phospholipid are the CCAAT/enhancer binding protein (C/EBP), monoglyceride lipase (Mgll), Nuclear Factor-kappaB activating protein (NF-κB AP-1) and Tnf receptor-associated factor 6 (Traf6). C/EBP plays a key role in acute-phase response to inflammatory cytokine IL-6 [34], Traf6 positively regulates the biosynthesis of interleukin-6 and interleukin-12, as well as the I-kappaB kinase/NF-kappaB cascade [35] and NF-κB AP-1 induce the expression of genes involved in inflammation [36]. Recent research suggests that brain inflammation may be the underlying cause of several cognitive disorders including ASD, aged associated cognitive decline and Alzheimer's disease. Alzheimer's disease is the most common cause of dementia and characterized clinically by progressive cognitive deterioration together with declining activities of daily living and neuropsychiatric symptoms or behavioral changes. Other embodiments of the invention are to use omega-3 rich phospholipids to reduce the symptoms of ASD, aged associated cognitive decline, aged associated memory decline and Alzheimer's disease. Non-limiting examples of the symptoms cognitive decline and Alzheimer's disease are memory loss, forgetfulness, short-term memory loss, aphasia, disorientation, disinhibition, behavioral changes and deterioration of musculature and mobility.

This invention also discloses that the fatty acid composition of the brain lipids and phospholipids changes after in take of omega-3 fatty acids for 30 days. A significant reduction of the arachidonic acids can be found in the phospholipids in the brain for the rats given either the EPA- or DRA-rich PL diets. This may affect the inflammatory response in this tissue and thereby have a great impact on cognitive diseases/conditions such as Parkinson's or and Alzheimer's where the inflammatory component is fundamental for the progression of the disease. This invention also discloses that the reduction of ARA is present also in the sn-2 position of the phospholipids in the brain. This is very important as the pro-inflammatory eicosanoids are produced from ARA, which are catalytically hydrolyzed from position 2 on the phospholipid by the action of phospholipase A2. The phospholipase A2 is released after stimuli at the cell wall, it then moves to the nuclear membrane where the hydrolysis of the phospholipid takes place.

Previously, it has been disclosed that omega-3 supplementation has an effect on learning ability in aged Wistar rats [37], as well as improving of retinal function [38] and trainability in puppies [39-42]. In addition it has been shown that extracted phospholipids from pig brain can enhance behavior, learning ability and retinal function in old mice [43].

In another embodiment of this invention, omega-3 rich phospholipids are utilized to improved cognitive and/or retinal function in mammals, preferably aging mammals, such as humans, and pets as dog and cats. Aging humans are generally older than about 40, 50, 60, 70 or 80 years old, while aging pets are preferably more than 5, 6, 7, 8, 9, 10, 11, or 12 years old. In some preferred embodiments, phospholipid compositions comprising EPA/DHA in ratio of from about 2:1 are administered in order to improve cognitive function and retinal function. Cognitive function is assessed using the delayed non-matching-to position task (DNMP). The task specifically requires dogs to remember the location of an object for either a short or long delay-period. The test assesses both general cognitive ability, which is indicated by overall performance and memory capacity, which is indicated by performance at long-delays. DNMP is highly sensitive to age and models the early deficits in visuospatial memory developing in mild cognitive impairment as in Alzheimer's disease [44]. The cognitive test data revealed statistically significant differences, with the low dose subjects doing better on the last 5 treatment sessions than they did in baseline testing or on the first five treatment sessions. By contrast, the high dose subjects performed more poorly at the long-delay on the last 5 treatment sessions. In addition, at the short delay, the low and medium dose groups showed substantial improvement on the last test block. These results suggest that the test compound can either improve or impair memory, depending on dose, and that the optimal dose is in the 12 to 26 mg/kg range. The fact that the greatest effect was observed at the short delay is evidence that the treatment produces a global improvement in cognitive functioning, rather than a selective improvement in memory. The ERG analysis revealed statistically significant increases in signal amplitude in the second component of the ERG response in the dark-adapted eye and statistically significant decrease in response latency. The most consistent effect in ERG was an increase in the amplitude of the scoptopic B wave response, which was observed at all three stimulus levels. The scoptopic response is the response of the dark adapted eye and is linked to the function of the rods, photoreceptors in the retina of the eye. The B wave response represents the response of post-receptor cells in the retina, predominantly, the bipolar and horizontal cells. The increased B-wave response, therefore, represents enhanced transmission of visual information from the photoreceptors to the second level retinal cells. In this instance, the enhanced response is for dark vision, but the canine eye contains a higher proportion of rods than that of the primate, suggesting that rod vision is of more general importance for the dog than the human. These results are consistent with the suggestion that retinal processing is enhanced by treatment with the test compound.

In some embodiments, the marine phospholipid compositions are derived from marine organisms such as fish, fish eggs, shrimp, krill, etc. In some embodiments, the marine phospholipids comprise a mixture of phosphatidylserine (PS), phosphatidylcholine (PC), phosphatidyl inositol (PI), and phosphatidylethanolamine (PE). Indeed, the present invention presents the surprising results that phospholipid compositions comprising a mixture of PC, PS, PI and PE are bioavailable and bioefficient. This results in an important advantage over phospholipid compositions synthesized or containing, for example, pure PS, PC, or PI which can be expensive and difficult to make. In some embodiments, the methods of the present invention utilize novel marine lipid compositions comprising an omega-3 containing phospholipid and a triacylglyceride (TG) in a ratio from about 1:10 to 10:1. Preferably the ratio is in the range of from about 3:1 to 1:3, more preferably the ratio is in the range of about 1:2 to 2:1. Preferably, the TG is a fish oil such as tuna oil, herring oil, menhaden oil, krill oil, cod liver oil or algae oil. However, this invention is not limited to omega-3 containing oils as other TG sources are contemplated such as vegetable oils. In some embodiments, the phospholipids in the composition have the following structure: 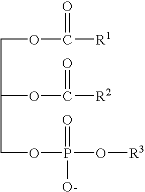
wherein R1 is OH or a fatty acid, R2 is OH or a fatty acid, and R3 is a mixture of H, choline, ethanolamine, inositol and serine. Attached to position 1 or position 2 are least 1% omega-3 fatty acids, preferably at least 5%, more preferably at least 10% omega-3 fatty acids, up to about 15%, 20%, 30%, 40%, 50%, or 60% omega-3 fatty acids. The omega-3 fatty acids can be EPA, DHA, DPA or C18:3 (n-3), most preferably the omega-3 fatty acids are EPA and DHA. The phospholipid composition preferably contains OH in position 1 or position 2 in a range of 25% to 50% in order to maximize absorption in-vivo.

Transesterification of phosphatidylcholine (PC) under solvent free conditions has been performed by Haraldsson et al in 1999 [15], with the results of high incorporation of EPA/DHA and with the following hydrolysis profile PC/LPC/GPC=39/44/17. Extensive hydrolysis and by-product formation is generally considered a problem with transesterification reactions, resulting in low product yields. This invention discloses a process for transesterification of crude soybean lecithin (mixture of PC, PE and PD). In the first step, the lecithin is hydrolyzed using a lipase in the presence of water (pH=8). The use of a variety of lipases is contemplated, including, but not limited to, Thermomyces Lanuginosus lipase, Rhizomucor miehei lipase, Candida Antarctica lipase, Pseudomonas fluorescence lipase, and Mucor javanicus lipase. The first step takes around 24 hours and results in a product comprising predominantly of lyso-phospholipids and glycerophospholipids such as PC/LPC/GPC=0/15/85. In the second step, free fatty acids are added such as EPA and DHA, however any omega-3 fatty acid is contemplated. Next a strong vacuum is applied to the reaction vessel for 72 hours. However, the reaction length can be varied in order to obtain a composition with the desired amount of phospholipids and lyso-phospholipids. By extending the reaction time beyond 72 hours, a product comprising more than 65% phospholipids can be obtained. Next, a lipid carrier is added to the reaction mixture in order to reduce the viscosity of the solution. The added amount of triglycerides can be 10%, 20%, 30%, 40% or more, it depends on the requested viscosity of the final product. The lipid carrier can be a fish oil such as tuna oil, menhaden oil and herring oil, or any triglyceride, diglyceride, ethyl- or methylester of a fatty acid. In the final step, the product is subjected to a molecular distillation and the free fatty acids are removed, resulting in a final product comprising of phospholipids (lyso-phospholipids and phospholipids) and triglycerides in a ratio of preferably 2:1.

In some embodiments, the compositions of this invention are contained in acceptable excipients and/or carriers for oral consumption. The actual form of the carrier, and thus, the compositions itself, is not critical. The carrier may be a liquid, gel, gelcap, capsule, powder, solid tablet (coated or non-coated), tea, or the like. The composition is preferably in the form of a tablet or capsule and most preferably in the form of a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In other embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The compositions of the present invention may also be formulated with a number of other compounds. These compounds and substances add to the palatability or sensory perception of the particles (e.g., flavorings and colorings) or improve the nutritional value of the particles (e.g., minerals, vitamins, phytonutrients, antioxidants, etc.).

### EXAMPLE 1

50g of soy lecithin from American Lecithin Company Inc (Oxford, CT, USA), 40g of TL-IM lipase from Novozymes (Bagsvaerd, Denmark) and 5g of water (adjusted to pH=8 using NaOH) were mixed in a reaction vessel at 50° C. for 24 hours. Next, 10 g of free fatty acids containing 10% EPA and 50% DHA from Napro Pharma (Brattvaag, Norway) was added, followed by application of vacuum to the reaction vessel. After 72 hours the reaction was terminated and the phospholipid mixture was analyzed using HPLC and GC. The results showed that the relationship between PC/LPC/GPC was 65/35/0, and that the content of EPA and DHA was around 10% and 12%, respectively. Next, 20g of sardine oil was added to the reaction mixture which comprised of 18% EPA and 12% DHA (relative GC peak area), followed by molecular distillation. The final product contained around 70% acetone insolubles, around 30% triglycerides and traces of free fatty acids.

### EXAMPLE 2

Marine phospholipids were prepared using either 40% soy PC (American Lecithin Company Inc, Oxford, CT, USA) (MPL1) according to the method in example 1 or using 96% pure soy PC (Phospholipid GmbH, Koln, Germany) (MPL2) according to a method described in [4]. Fatty acid content and the level of bi-products are shown in table 1. The MPL treatments consisted of a mixture of phospholipids, lyso-phospholipids and glycerol-phospholipids. Looking only at the PC/LPC/GPC relationship, it was 64/33/2 and 42/40/18 for MPL1 and MPL2, respectively. Finally, all three treatments were emulsified into skimmed milk.

TABLE 1
Composition of the phospholipids used in example 1

| | PC/ | | | | |
|---|---|---|---|---|---|
| Composition | LPC/GPC | 18:2 (n-6) | 18:3 (n-3) | EPA | DHA |
| MPL1 | 64/33/2 | 129 mg/g | 9 mg/g | 51 mg/g | 171 mg/g |
| MPL2 | 42/40/18 | 124 mg/g | 9 mg/g | 96 mg/g | 96 mg/g |

18 newly weaned Sprague-Dawley rats were fed the milk emulsions for 1 week. Each rat was placed in its own cage to ensure that they got an even amount of test substance and the milk was consumed by the rat pups ad libitum. After 1 week the experiment was terminated and the rats were decapitated. The animals were kept without food for 24 hours before sampling. Entire livers were collected and frozen immediately using liquid nitrogen (stored at −65° C.). Total RNA was isolated from the liver samples according to the Quiagen Rnaesy Midi Kit Protocol. The RNA samples were quantified and quality measured by NanoDrop and Bioanalyzer. The isolated RNA was hybridized onto a gene chip RAE230 2.0 from Affymetrix (Santa Clara, Calif., USA). The expression level of each gene was measured using an Affymetrix GeneChip 3000 7G scanner. The results were suitable for all chips except 2 and they were excluded from the trial. The results are based on (log) probe set summary expression measures, computed by RMA, and linear models are fitted using Empirical Bayes methods for borrowing strength across genes (using the Limma package in R). The p-value are adjusted for multiple testing using the Benjamini-Hochberg-method, controlling the False Discovery Rate (FDR), where FDR=the proportion of null-hypotheses of no DE that are falsely rejected.

MPL2 regulates 401 genes versus the control (table 2). A number of genes listed are involved maintenance of the cell, in transcription and protein synthesis as well as signaling pathways. Others are involved in regulation of metabolism and the inflammatory response such as Tnf receptor-associated factor 6 (Traf6_predicted) (fold change of 0.53), guanine nucleotide binding protein alpha inhibiting 2 (Gnai2) (fold change of 0.6, gamma-butyrobetaine hydroxylase (Bbox1) (fold change of 1.32), monoglyceride lipase (Mgll) (fold change 0.52), nuclear NF-kappaB activating protein (fold change 0.65) and CCAAT/enhancer binding protein (C/EBP) (fold change of 0.66).

TABLE 2
List of genes differentially expressed (DE) by MPL2 versus control.
SLR: Estimated signal log-ratio (<0: down regulated gene,
>0: up regulated gene).
Fold change: Estimated fold change corresponding
to the parameter (<1: down regulated gene, >1: up
regulated gene). Affy fold change: Estimated fold change
using the Affymetrix definition (<−1: down regulated gene,
>1: up regulated gene) df: Degrees of freedom (=number of arrays − number of estimated parameters)

| | | | | | | Fold | Affy | |
|---|---|---|---|---|---|---|---|---|
| Gene-ID | Gene name | t | p-value | FDR | SLR | change | Fold. change | df |
| 1367588_a_at | ribosomal protein L13A | −5.22 | 0.00005 | 0.00568 | −0.44 | 0.74 | −1.36 | 14 |
| 1367844_at | guanine nucleotide binding | −5.47 | 0.00003 | 0.00417 | −0.54 | 0.69 | −1.46 | 14 |
| | protein, alpha inhibiting 2 | | | | | | | |
| 1367958_at | abl-interactor 1 | −6.42 | 0.00000 | 0.00119 | −0.69 | 0.62 | −1.62 | 14 |
| 1367971_at | protein tyrosine phosphatase | −6.01 | 0.00001 | 0.00190 | −0.36 | 0.78 | −1.28 | 14 |
| | 4a2 | | | | | | | |
| 1368057_at | ATP-binding cassette, sub- | −5.06 | 0.00007 | 0.00688 | −0.54 | 0.69 | −1.45 | 14 |
| | family D (ALD), member 3 | | | | | | | |
| 1368405_at | v-ral simian leukemia viral | −4.86 | 0.00011 | 0.00913 | −0.44 | 0.74 | −1.36 | 14 |
| | oncogene homolog A (ras | | | | | | | |
| | related) | | | | | | | |
| 1368646_at | mitogen-activated protein | 4.98 | 0.00008 | 0.00772 | 0.70 | 1.62 | 1.62 | 14 |
| | kinase 9 | | | | | | | |
| 1368649_at | dyskeratosis congenita 1, | −6.73 | 0.00000 | 0.00080 | −0.53 | 0.69 | −1.44 | 14 |
| | dyskerin | | | | | | | |
| 1368662_at | ring finger protein 39 | −7.01 | 0.00000 | 0.00053 | −0.61 | 0.66 | −1.52 | 14 |
| 1368703_at | enigma homolog | −5.38 | 0.00003 | 0.00450 | −0.76 | 0.59 | −1.70 | 14 |
| 1368824_at | caldesmon 1 | −7.18 | 0.00000 | 0.00043 | −1.00 | 0.50 | −2.00 | 14 |
| 1368841_at | transcription factor 4 | −4.94 | 0.00009 | 0.00828 | −0.38 | 0.77 | −1.31 | 14 |
| 1368867_at | GERp95 | −7.83 | 0.00000 | 0.00019 | −0.85 | 0.56 | −1.80 | 14 |
| 1369094_a_at | protein tyrosine phosphatase, | −7.22 | 0.00000 | 0.00042 | −0.97 | 0.51 | −1.96 | 14 |
| | receptor type, D | | | | | | | |
| 1369127_a_at | prostaglandin F receptor | 4.85 | 0.00011 | 0.00921 | 0.45 | 1.37 | 1.37 | 14 |
| 1369174_at | SMAD, mothers against DPP | −5.19 | 0.00005 | 0.00581 | −0.38 | 0.77 | −1.30 | 14 |
| | homolog 1 (Drosophila) | | | | | | | |
| 1369227_at | Choroidermia | 5.04 | 0.00007 | 0.00718 | 0.47 | 1.39 | 1.39 | 14 |
| 1369249_at | progressive ankylosis homolog | 5.36 | 0.00004 | 0.00467 | 0.48 | 1.39 | 1.39 | 14 |
| | (mouse) | | | | | | | |
| 1369501_at | zinc finger protein 260 | 5.17 | 0.00005 | 0.00595 | 0.41 | 1.33 | 1.33 | 14 |
| 1369517_at | pleckstrin homology, Sec7 and | 4.93 | 0.00009 | 0.00829 | 0.48 | 1.40 | 1.40 | 14 |
| | coiled/coil domains 1 | | | | | | | |
| 1369546_at | butyrobetaine (gamma), 2- | 4.96 | 0.00009 | 0.00811 | 0.40 | 1.32 | 1.32 | 14 |
| | oxoglutarate dioxygenase 1 | | | | | | | |
| | (gamma-butyrobetaine | | | | | | | |
| | hydroxylase) | | | | | | | |
| 1369628_at | synaptic vesicle glycoprotein | −7.20 | 0.00000 | 0.00042 | −1.11 | 0.46 | −2.15 | 14 |
| | 2b | | | | | | | |
| 1369689_at | N-ethylmaleimide sensitive | 6.22 | 0.00001 | 0.00155 | 0.66 | 1.58 | 1.58 | 14 |
| | fusion protein | | | | | | | |
| 1369736_at | epithelial membrane protein 1 | 5.74 | 0.00002 | 0.00286 | 0.62 | 1.54 | 1.54 | 14 |
| 1369775_at | nuclear ubiquitous casein | −7.56 | 0.00000 | 0.00027 | −0.79 | 0.58 | −1.73 | 14 |
| | kinase and cyclin-dependent | | | | | | | |
| | kinase substrate | | | | | | | |
| 1370184_at | cofilin 1 | −6.07 | 0.00001 | 0.00178 | −0.38 | 0.77 | −1.30 | 14 |
| 1370260_at | adducin 3 (gamma) | −5.50 | 0.00003 | 0.00399 | −0.76 | 0.59 | −1.70 | 14 |
| 1370328_at | Dickkopf homolog 3 (Xenopus | 4.80 | 0.00012 | 0.00964 | 0.59 | 1.51 | 1.51 | 14 |
| | laevis) | | | | | | | |
| 1370717_at | AP1 gamma subunit binding | 6.00 | 0.00001 | 0.00192 | 0.58 | 1.50 | 1.50 | 14 |
| | protein 1 | | | | | | | |
| 1370831_at | monoglyceride lipase | −5.47 | 0.00003 | 0.00414 | −0.94 | 0.52 | −1.92 | 14 |
| 1370901_at | similar to hypothetical protein | −4.83 | 0.00012 | 0.00948 | −0.34 | 0.79 | −1.27 | 14 |
| | MGC36831 (predicted) | | | | | | | |
| 1370946_at | nuclear factor I/X | −10.64 | 0.00000 | 0.00002 | −1.17 | 0.45 | −2.25 | 14 |
| 1370949_at | myristoylated alanine rich | −7.58 | 0.00000 | 0.00026 | −1.17 | 0.44 | −2.26 | 14 |
| | protein kinase C substrate | | | | | | | |
| 1370993_at | laminin, gamma 1 | 6.13 | 0.00001 | 0.00171 | 0.63 | 1.54 | 1.54 | 14 |
| 1371034_at | one cut domain, family | −5.65 | 0.00002 | 0.00327 | −1.77 | 0.29 | −3.40 | 14 |
| | member 1 | | | | | | | |
| 1371059_at | protein kinase, cAMP- | 5.24 | 0.00005 | 0.00556 | 0.48 | 1.40 | 1.40 | 14 |
| | dependent, regulatory, type 2, | | | | | | | |
| | alpha | | | | | | | |
| 1371345_at | methyl-CpG binding domain | −5.32 | 0.00004 | 0.00491 | −0.34 | 0.79 | −1.27 | 14 |
| | protein 3 (predicted) | | | | | | | |
| 1371361_at | similar to tensin | −7.21 | 0.00000 | 0.00042 | −0.60 | 0.66 | −1.51 | 14 |
| 1371394_x_at | similar to Ab2-143 | −5.11 | 0.00006 | 0.00645 | −0.63 | 0.64 | −1.55 | 14 |
| 1371397_at | nitric oxide synthase | −5.53 | 0.00002 | 0.00383 | −0.34 | 0.79 | −1.26 | 14 |
| | interacting protein (predicted) | | | | | | | |
| 1371428_at | | −5.76 | 0.00001 | 0.00276 | −0.37 | 0.77 | −1.29 | 14 |
| 1371430_at | dystroglycan 1 | −5.46 | 0.00003 | 0.00417 | −0.62 | 0.65 | −1.53 | 14 |
| 1371432_at | | −4.95 | 0.00009 | 0.00811 | −0.36 | 0.78 | −1.28 | 14 |
| 1371452_at | bone marrow stromal cell- | −5.05 | 0.00007 | 0.00705 | −0.46 | 0.73 | −1.37 | 14 |
| | derived ubiquitin-like protein | | | | | | | |
| 1371573_at | ribosomal protein L36a | −5.90 | 0.00001 | 0.00221 | −0.40 | 0.76 | −1.32 | 14 |
| | (predicted) | | | | | | | |
| 1371589_at | Ubiquitin-Like 5 Protein | −5.28 | 0.00004 | 0.00518 | −0.57 | 0.68 | −1.48 | 14 |
| 1371590_s_at | Ubiquitin-Like 5 Protein | −4.94 | 0.00009 | 0.00829 | −0.39 | 0.76 | −1.31 | 14 |
| 1371779_at | sorting nexin 6 (predicted) | 5.64 | 0.00002 | 0.00329 | 0.63 | 1.55 | 1.55 | 14 |
| 1371826_at | Transcribed locus | −5.58 | 0.00002 | 0.00359 | −0.48 | 0.72 | −1.39 | 14 |
| 1371896_at | growth arrest and DNA- | −6.02 | 0.00001 | 0.00189 | −0.43 | 0.74 | −1.35 | 14 |
| | damage-inducible, gamma | | | | | | | |
| | interacting protein 1 (predicted) | | | | | | | |
| 1371918_at | CD99 | −5.35 | 0.00004 | 0.00476 | −0.37 | 0.77 | −1.29 | 14 |
| 1372057_at | CDNA clone MGC: 124976 | −6.12 | 0.00001 | 0.00173 | −0.38 | 0.77 | −1.30 | 14 |
| | IMAGE: 7110947 | | | | | | | |
| 1372137_at | biogenesis of lysosome-related | −6.03 | 0.00001 | 0.00187 | −0.41 | 0.75 | −1.32 | 14 |
| | organelles complex-1, subunit | | | | | | | |
| | 1 (predicted) | | | | | | | |
| 1372142_at | arsA arsenite transporter, ATP- | −4.93 | 0.00009 | 0.00829 | −0.37 | 0.77 | −1.30 | 14 |
| | binding, homolog 1 (bacterial) | | | | | | | |
| | (predicted) | | | | | | | |
| 1372236_at | Similar to Caspase recruitment | −4.90 | 0.00010 | 0.00871 | −0.36 | 0.78 | −1.29 | 14 |
| | domain protein 4 | | | | | | | |
| 1372469_at | Transcribed locus | −4.84 | 0.00011 | 0.00945 | −0.36 | 0.78 | −1.28 | 14 |
| 1372697_at | mitochondrial ribosomal | −5.70 | 0.00002 | 0.00299 | −0.58 | 0.67 | −1.49 | 14 |
| | protein S15 | | | | | | | |
| 1373031_at | tripartite motif protein 8 | −5.13 | 0.00006 | 0.00628 | −0.44 | 0.74 | −1.36 | 14 |
| | (predicted) | | | | | | | |
| 1373105_at | interleukin 1 receptor-like 1 | −5.01 | 0.00008 | 0.00742 | −0.37 | 0.77 | −1.30 | 14 |
| | ligand (predicted) | | | | | | | |
| 1373135_at | similar to hypothetical protein | −5.30 | 0.00004 | 0.00503 | −0.55 | 0.68 | −1.46 | 14 |
| | MGC2744 | | | | | | | |
| 1373206_at | similar to FAD104 (predicted) | 6.73 | 0.00000 | 0.00080 | 0.64 | 1.56 | 1.56 | 14 |
| 1373303_at | similar to mKIAA3013 protein | −5.28 | 0.00004 | 0.00514 | −0.48 | 0.72 | −1.39 | 14 |
| 1373347_at | DMT1-associated protein | −6.18 | 0.00001 | 0.00162 | −0.73 | 0.60 | −1.66 | 14 |
| 1373378_at | ATP/GTP binding protein 1 | 5.39 | 0.00003 | 0.00449 | 0.51 | 1.42 | 1.42 | 14 |
| | (predicted) | | | | | | | |
| 1373804_at | Forkhead box P1 (predicted) | −5.28 | 0.00004 | 0.00518 | −0.59 | 0.66 | −1.51 | 14 |
| 1373885_at | chromobox homolog 5 | −5.94 | 0.00001 | 0.00208 | −1.04 | 0.48 | −2.06 | 14 |
| | (Drosophila HP1a) (predicted) | | | | | | | |
| 1374002_at | | −6.78 | 0.00000 | 0.00074 | −0.86 | 0.55 | −1.82 | 14 |
| 1374283_at | fetal Alzheimer antigen | −7.44 | 0.00000 | 0.00032 | −0.74 | 0.60 | −1.67 | 14 |
| | (predicted) | | | | | | | |
| 1374425_at | transducin-like enhancer of | −4.91 | 0.00010 | 0.00849 | −0.40 | 0.76 | −1.32 | 14 |
| | split 1, homolog of Drosophila | | | | | | | |
| | E(spl) (predicted) | | | | | | | |
| 1374509_at | Similar to RIKEN cDNA | −5.62 | 0.00002 | 0.00337 | −0.47 | 0.72 | −1.39 | 14 |
| | 1110018O08 | | | | | | | |
| 1374511_at | | 5.60 | 0.00002 | 0.00345 | 0.55 | 1.47 | 1.47 | 14 |
| 1374657_at | Transcribed locus | −4.88 | 0.00010 | 0.00890 | −0.34 | 0.79 | −1.27 | 14 |
| 1374733_at | symplekin (predicted) | −5.04 | 0.00007 | 0.00716 | −0.36 | 0.78 | −1.28 | 14 |
| 1374772_at | similar to Chromosome 13 | 5.18 | 0.00005 | 0.00581 | 0.46 | 1.38 | 1.38 | 14 |
| | open reading frame 21 | | | | | | | |
| 1374837_at | B-cell CLL/lymphoma 7C | −8.92 | 0.00000 | 0.00006 | −0.71 | 0.61 | −1.63 | 14 |
| | (predicted) | | | | | | | |
| 1374851_at | similar to RIKEN cDNA | −4.89 | 0.00010 | 0.00879 | −0.39 | 0.76 | −1.31 | 14 |
| | 2810405O22 (predicted) | | | | | | | |
| 1374852_at | hypothetical LOC362592 | −5.20 | 0.00005 | 0.00579 | −0.37 | 0.78 | −1.29 | 14 |
| 1375214_at | UDP-N-acetyl-alpha-D- | −5.31 | 0.00004 | 0.00500 | −0.58 | 0.67 | −1.50 | 14 |
| | galactosamine:polypeptide N- | | | | | | | |
| | acetylgalactosaminyltransferase | | | | | | | |
| | 2 (predicted) | | | | | | | |
| 1375335_at | heat shock 90 kDa protein 1, | −5.26 | 0.00004 | 0.00538 | −0.55 | 0.68 | −1.46 | 14 |
| | beta | | | | | | | |
| 1375396_at | pumilio 1 (Drosophila) | −10.05 | 0.00000 | 0.00003 | −0.92 | 0.53 | −1.89 | 14 |
| | (predicted) | | | | | | | |
| 1375421_a_at | praja 2, RING-H2 motif | −6.51 | 0.00000 | 0.00102 | −0.60 | 0.66 | −1.52 | 14 |
| | containing | | | | | | | |
| 1375453_at | | −12.32 | 0.00000 | 0.00000 | −1.02 | 0.49 | −2.02 | 14 |
| 1375469_at | SWI/SNF related, matrix | −7.97 | 0.00000 | 0.00017 | −0.93 | 0.53 | −1.90 | 14 |
| | associated, actin dependent | | | | | | | |
| | regulator of chromatin, | | | | | | | |
| | subfamily a, member 4 | | | | | | | |
| 1375533_at | vestigial like 4 (Drosophila) | −5.30 | 0.00004 | 0.00505 | −0.61 | 0.66 | −1.52 | 14 |
| | (predicted) | | | | | | | |
| 1375548_at | similar to RIKEN cDNA | −5.64 | 0.00002 | 0.00328 | −0.58 | 0.67 | −1.50 | 14 |
| | 4732418C07 (predicted) | | | | | | | |
| 1375621_at | | −7.05 | 0.00000 | 0.00051 | −0.96 | 0.51 | −1.95 | 14 |
| 1375632_at | similar to 60S ribosomal | −4.85 | 0.00011 | 0.00921 | −0.29 | 0.82 | −1.22 | 14 |
| | protein L38 | | | | | | | |
| 1375650_at | bromodomain containing 4 | −6.64 | 0.00000 | 0.00088 | −0.48 | 0.71 | −1.40 | 14 |
| | (predicted) | | | | | | | |
| 1375658_at | Transcribed locus | −5.00 | 0.00008 | 0.00756 | −0.44 | 0.74 | −1.35 | 14 |
| 1375696_at | interferon (alpha and beta) | 4.81 | 0.00012 | 0.00958 | 0.59 | 1.51 | 1.51 | 14 |
| | receptor 1 (predicted) | | | | | | | |
| 1375703_at | myeloid/lymphoid or mixed- | −10.20 | 0.00000 | 0.00003 | −1.02 | 0.49 | −2.03 | 14 |
| | lineage leukemia 5 (trithorax | | | | | | | |
| | homolog, Drosophila) | | | | | | | |
| | (predicted) | | | | | | | |
| 1375706_at | | −5.01 | 0.00008 | 0.00743 | −0.49 | 0.71 | −1.40 | 14 |
| 1375763_at | similar to 2700008B19Rik | −7.08 | 0.00000 | 0.00050 | −0.54 | 0.69 | −1.45 | 14 |
| | protein | | | | | | | |
| 1375958_at | | −5.13 | 0.00006 | 0.00628 | −0.65 | 0.64 | −1.57 | 14 |
| 1376059_at | | 5.33 | 0.00004 | 0.00483 | 0.35 | 1.28 | 1.28 | 14 |
| 1376256_at | WD repeat and FYVE domain | −9.16 | 0.00000 | 0.00005 | −1.10 | 0.47 | −2.15 | 14 |
| | containing 1 (predicted) | | | | | | | |
| 1376299_at | similar to Retinoblastoma- | −9.22 | 0.00000 | 0.00005 | −0.89 | 0.54 | −1.85 | 14 |
| | binding protein 2 (RBBP-2) | | | | | | | |
| 1376450_at | transmembrane protein 5 | −6.26 | 0.00001 | 0.00147 | −0.55 | 0.68 | −1.46 | 14 |
| | (predicted) | | | | | | | |
| 1376523_at | AT rich interactive domain 4A | −5.53 | 0.00002 | 0.00383 | −0.77 | 0.59 | −1.70 | 14 |
| | (Rbp1 like) (predicted) | | | | | | | |
| 1376524_at | hypothetical protein Dd25 | −6.69 | 0.00000 | 0.00082 | −0.66 | 0.63 | −1.58 | 14 |
| 1376532_at | similar to FAD104 (predicted) | 6.06 | 0.00001 | 0.00178 | 0.56 | 1.47 | 1.47 | 14 |
| 1376728_at | Transcribed locus | −4.80 | 0.00012 | 0.00966 | −0.35 | 0.78 | −1.27 | 14 |
| 1376917_at | zinc finger protein 292 | −5.21 | 0.00005 | 0.00571 | −0.66 | 0.63 | −1.58 | 14 |
| 1376982_at | Transcribed locus | −5.49 | 0.00003 | 0.00405 | −0.45 | 0.73 | −1.37 | 14 |
| 1377105_at | | −6.97 | 0.00000 | 0.00056 | −0.89 | 0.54 | −1.85 | 14 |
| 1377302_a_at | methylmalonic aciduria | −5.10 | 0.00006 | 0.00660 | −0.52 | 0.70 | −1.43 | 14 |
| | (cobalamin deficiency) type A | | | | | | | |
| | (predicted) | | | | | | | |
| 1377524_at | similar to CG18661-PA | −5.36 | 0.00003 | 0.00465 | −0.43 | 0.74 | −1.35 | 14 |
| | (predicted) | | | | | | | |
| 1377663_at | ras homolog gene family, | −5.00 | 0.00008 | 0.00756 | −0.87 | 0.55 | −1.82 | 14 |
| | member E | | | | | | | |
| 1377683_at | similar to hypothetical protein | −6.63 | 0.00000 | 0.00088 | −0.56 | 0.68 | −1.47 | 14 |
| | FLJ13045 (predicted) | | | | | | | |
| 1377728_at | LOC499567 | −5.45 | 0.00003 | 0.00419 | −1.03 | 0.49 | −2.04 | 14 |
| 1377766_at | Transcribed locus | 4.80 | 0.00012 | 0.00964 | 0.37 | 1.29 | 1.29 | 14 |
| 1377899_at | similar to RIKEN cDNA | −4.99 | 0.00008 | 0.00760 | −0.46 | 0.73 | −1.38 | 14 |
| | 2810025M15 (predicted) | | | | | | | |
| 1377906_at | DEAH (Asp-Glu-Ala-His) box | −4.82 | 0.00012 | 0.00950 | −0.73 | 0.60 | −1.66 | 14 |
| | polypeptide 36 (predicted) | | | | | | | |
| 1377914_at | serine/arginine repetitive | −6.41 | 0.00000 | 0.00120 | −0.98 | 0.51 | −1.97 | 14 |
| | matrix 1 (predicted) | | | | | | | |
| 1378155_at | similar to KIAA1096 protein | −5.68 | 0.00002 | 0.00313 | −0.89 | 0.54 | −1.86 | 14 |
| 1378163_at | Transcribed locus | −4.86 | 0.00011 | 0.00913 | −0.78 | 0.58 | −1.71 | 14 |
| 1378170_at | Transcribed locus | −5.00 | 0.00008 | 0.00756 | −0.92 | 0.53 | −1.90 | 14 |
| 1378194_a_at | rap2 interacting protein x | −4.82 | 0.00012 | 0.00950 | −0.72 | 0.61 | −1.65 | 14 |
| 1378361_at | chromodomain helicase DNA | −7.32 | 0.00000 | 0.00039 | −0.73 | 0.60 | −1.66 | 14 |
| | binding protein 7 (predicted) | | | | | | | |
| 1378453_at | | −4.84 | 0.00011 | 0.00938 | −0.74 | 0.60 | −1.66 | 14 |
| 1378504_at | Insulin-like growth factor I | −5.41 | 0.00003 | 0.00440 | −0.96 | 0.51 | −1.95 | 14 |
| | mRNA, 3′ end of mRNA | | | | | | | |
| 1378786_at | Transcribed locus, weakly | 4.89 | 0.00010 | 0.00879 | 0.33 | 1.25 | 1.25 | 14 |
| | similar to NP_780607.2 | | | | | | | |
| | hypothetical protein | | | | | | | |
| | LOC109050 [Mus musculus] | | | | | | | |
| 1379062_at | similar to Expressed sequence | −6.60 | 0.00000 | 0.00090 | −1.08 | 0.47 | −2.12 | 14 |
| | AU019823 | | | | | | | |
| 1379073_at | Similar to RIKEN cDNA | −5.51 | 0.00003 | 0.00394 | −0.49 | 0.71 | −1.40 | 14 |
| | 2310067G05 | | | | | | | |
| 1379101_at | DEAH (Asp-Glu-Ala-His) box | −5.55 | 0.00002 | 0.00375 | −0.87 | 0.55 | −1.82 | 14 |
| | polypeptide 36 (predicted) | | | | | | | |
| 1379112_at | AT rich interactive domain 4A | −5.70 | 0.00002 | 0.00299 | −0.44 | 0.74 | −1.35 | 14 |
| | (Rbp1 like) (predicted) | | | | | | | |
| 1379232_at | TBC1D12: TBC1 domain | −6.98 | 0.00000 | 0.00056 | −1.40 | 0.38 | −2.63 | 14 |
| | family, member 12 (predicted) | | | | | | | |
| 1379330_s_at | CDNA clone IMAGE: 7316839 | −4.80 | 0.00012 | 0.00967 | −0.36 | 0.78 | −1.28 | 14 |
| 1379332_at | Transcribed locus, strongly | −4.88 | 0.00010 | 0.00886 | −0.61 | 0.66 | −1.52 | 14 |
| | similar to XP_417265.1 | | | | | | | |
| | PREDICTED: similar to F- | | | | | | | |
| | box-WD40 repeat protein 6 | | | | | | | |
| | [Gallus gallus] | | | | | | | |
| 1379399_at | similar to cDNA sequence | −5.37 | 0.00003 | 0.00459 | −0.42 | 0.75 | −1.34 | 14 |
| | BC016188 (predicted) | | | | | | | |
| 1379457_at | neural precursor cell expressed, | −5.39 | 0.00003 | 0.00449 | −0.56 | 0.68 | −1.48 | 14 |
| | developmentally down- | | | | | | | |
| | regulated gene 1 (predicted) | | | | | | | |
| 1379469_at | similar to transducin (beta)-like | −6.23 | 0.00001 | 0.00153 | −0.91 | 0.53 | −1.88 | 14 |
| | 1 X-linked | | | | | | | |
| 1379485_at | eukaryotic translation initiation | −7.08 | 0.00000 | 0.00050 | −1.68 | 0.31 | −3.21 | 14 |
| | factor 3, subunit 10 (theta) | | | | | | | |
| | (predicted) | | | | | | | |
| 1379571_at | plakophilin 4 (predicted) | −5.42 | 0.00003 | 0.00436 | −0.74 | 0.60 | −1.67 | 14 |
| 1379578_at | similar to Zbtb20 protein | −8.89 | 0.00000 | 0.00006 | −0.71 | 0.61 | −1.63 | 14 |
| 1379662_a_at | SNF related kinase | 4.93 | 0.00009 | 0.00829 | 0.36 | 1.29 | 1.29 | 14 |
| 1379715_at | similar to CG9346-PA | −4.93 | 0.00009 | 0.00829 | −0.71 | 0.61 | −1.63 | 14 |
| | (predicted) | | | | | | | |
| 1379826_at | similar to hypothetical protein | −5.95 | 0.00001 | 0.00208 | −0.62 | 0.65 | −1.54 | 14 |
| | MGC31967 | | | | | | | |
| 1380008_at | similar to Neurofilament triplet | −5.11 | 0.00006 | 0.00645 | −0.60 | 0.66 | −1.52 | 14 |
| | H protein (200 kDa | | | | | | | |
| | neurofilament protein) | | | | | | | |
| | (Neurofilament heavy | | | | | | | |
| | polypeptide) (NF-H) | | | | | | | |
| | (predicted) | | | | | | | |
| 1380060_at | DNA topoisomerase I, | −5.23 | 0.00005 | 0.00566 | −0.53 | 0.69 | −1.44 | 14 |
| | mitochondrial | | | | | | | |
| 1380062_at | membrane protein, | −6.88 | 0.00000 | 0.00065 | −0.75 | 0.59 | −1.68 | 14 |
| | palmitoylated 6 (MAGUK p55 | | | | | | | |
| | subfamily member 6) | | | | | | | |
| | (predicted) | | | | | | | |
| 1380166_at | Similar to hypothetical protein | 5.63 | 0.00002 | 0.00333 | 0.34 | 1.27 | 1.27 | 14 |
| | FLJ12056 | | | | | | | |
| 1380371_at | delangin (predicted) | −9.37 | 0.00000 | 0.00005 | −0.94 | 0.52 | −1.91 | 14 |
| 1380446_at | myeloid/lymphoid or mixed- | −5.00 | 0.00008 | 0.00756 | −0.62 | 0.65 | −1.54 | 14 |
| | lineage leukemia (trithorax | | | | | | | |
| | homolog, Drosophila); | | | | | | | |
| | translocated to, 10 (predicted) | | | | | | | |
| 1380503_at | hypothetical LOC305452 | −6.07 | 0.00001 | 0.00178 | −0.62 | 0.65 | −1.53 | 14 |
| | (predicted) | | | | | | | |
| 1380728_at | Similar to collapsin response | 6.09 | 0.00001 | 0.00178 | 0.49 | 1.41 | 1.41 | 14 |
| | mediator protein-2A | | | | | | | |
| 1381469_a_at | PERQ amino acid rich, with | −5.49 | 0.00003 | 0.00405 | −0.51 | 0.70 | −1.43 | 14 |
| | GYF domain 1 (predicted) | | | | | | | |
| 1381525_at | | −4.82 | 0.00012 | 0.00952 | −0.41 | 0.75 | −1.33 | 14 |
| 1381542_at | UBX domain containing 2 | −6.15 | 0.00001 | 0.00171 | −0.83 | 0.56 | −1.78 | 14 |
| | (predicted) | | | | | | | |
| 1381548_at | golgi phosphoprotein 4 | −5.81 | 0.00001 | 0.00256 | −0.69 | 0.62 | −1.61 | 14 |
| | (predicted) | | | | | | | |
| 1381567_at | hypothetical LOC294390 | 4.97 | 0.00008 | 0.00800 | 0.36 | 1.29 | 1.29 | 14 |
| | (predicted) | | | | | | | |
| 1381764_s_at | ring finger protein 126 | −5.54 | 0.00002 | 0.00382 | −0.51 | 0.70 | −1.42 | 14 |
| | (predicted) | | | | | | | |
| 1381809_at | ankyrin repeat domain 11 | −5.94 | 0.00001 | 0.00209 | −1.11 | 0.46 | −2.17 | 14 |
| | (predicted) | | | | | | | |
| 1381829_at | | −6.27 | 0.00000 | 0.00145 | −1.07 | 0.48 | −2.10 | 14 |
| 1381878_at | ubinuclein 1 (predicted) | −5.82 | 0.00001 | 0.00252 | −1.18 | 0.44 | −2.26 | 14 |
| 1381958_at | similar to mKIAA0259 protein | −6.90 | 0.00000 | 0.00062 | −1.27 | 0.41 | −2.42 | 14 |
| 1382000_at | | 4.82 | 0.00012 | 0.00950 | 0.41 | 1.33 | 1.33 | 14 |
| 1382009_at | Transcribed locus | −5.39 | 0.00003 | 0.00449 | −0.69 | 0.62 | −1.62 | 14 |
| 1382027_at | LOC498010 | −6.28 | 0.00000 | 0.00144 | −0.76 | 0.59 | −1.70 | 14 |
| 1382056_at | similar to splicing factor p54 | −8.13 | 0.00000 | 0.00016 | −0.97 | 0.51 | −1.96 | 14 |
| 1382109_at | nuclear NF-kappaB activating | −5.83 | 0.00001 | 0.00250 | −0.62 | 0.65 | −1.53 | 14 |
| | protein | | | | | | | |
| 1382155_at | | 6.37 | 0.00000 | 0.00126 | 0.58 | 1.50 | 1.50 | 14 |
| 1382193_at | Transcribed locus | −6.07 | 0.00001 | 0.00178 | −1.42 | 0.37 | −2.67 | 14 |
| 1382306_at | Ariadne ubiquitin-conjugating | 6.59 | 0.00000 | 0.00090 | 0.59 | 1.50 | 1.50 | 14 |
| | enzyme E2 binding protein | | | | | | | |
| | homolog 1 (Drosophila) | | | | | | | |
| | (predicted) | | | | | | | |
| 1382307_at | protein phosphatase 1, | −4.79 | 0.00013 | 0.00976 | −0.47 | 0.72 | −1.39 | 14 |
| | regulatory (inhibitor) subunit | | | | | | | |
| | 12A | | | | | | | |
| 1382358_at | Similar to SRY (sex | −5.34 | 0.00004 | 0.00482 | −0.65 | 0.64 | −1.57 | 14 |
| | determining region Y)-box 5 | | | | | | | |
| | isoform a | | | | | | | |
| 1382372_at | Aryl hydrocarbon receptor | −5.07 | 0.00007 | 0.00680 | −0.74 | 0.60 | −1.67 | 14 |
| 1382430_at | similar to KIAA1585 protein | −5.62 | 0.00002 | 0.00338 | −0.58 | 0.67 | −1.50 | 14 |
| | (predicted) | | | | | | | |
| 1382434_at | ectonucleoside triphosphate | −5.89 | 0.00001 | 0.00229 | −0.73 | 0.60 | −1.66 | 14 |
| | diphosphohydrolase 5 | | | | | | | |
| 1382466_at | similar to RIKEN cDNA | −5.43 | 0.00003 | 0.00433 | −0.98 | 0.51 | −1.97 | 14 |
| | 6530403A03 (predicted) | | | | | | | |
| 1382551_at | similar to Intersectin 2 (SH3 | −6.72 | 0.00000 | 0.00080 | −1.41 | 0.38 | −2.67 | 14 |
| | domain-containing protein 1B) | | | | | | | |
| | (SH3P18) (SH3P18-like | | | | | | | |
| | WASP associated protein) | | | | | | | |
| 1382558_at | transcription factor 3 | −6.13 | 0.00001 | 0.00171 | −0.62 | 0.65 | −1.54 | 14 |
| | (predicted) | | | | | | | |
| 1382573_at | Transcribed locus | 5.08 | 0.00007 | 0.00677 | 0.38 | 1.30 | 1.30 | 14 |
| 1382584_at | similar to mKIAA1321 protein | −7.22 | 0.00000 | 0.00042 | −1.15 | 0.45 | −2.22 | 14 |
| 1382620_at | ankyrin repeat domain 11 | −9.69 | 0.00000 | 0.00003 | −0.95 | 0.52 | −1.93 | 14 |
| | (predicted) | | | | | | | |
| 1382797_at | similar to 1500019C06Rik | −5.02 | 0.00008 | 0.00742 | −0.47 | 0.72 | −1.39 | 14 |
| | protein | | | | | | | |
| 1382813_at | similar to RIKEN cDNA | −5.36 | 0.00004 | 0.00467 | −0.46 | 0.73 | −1.38 | 14 |
| | 4930444A02 (predicted) | | | | | | | |
| 1382862_at | Transcribed locus | −6.23 | 0.00001 | 0.00153 | −1.16 | 0.45 | −2.23 | 14 |
| 1382904_at | similar to hypothetical protein | −9.04 | 0.00000 | 0.00005 | −0.85 | 0.56 | −1.80 | 14 |
| | DKFZp434K1421 (predicted) | | | | | | | |
| 1382935_at | similar to Hypothetical protein | −6.54 | 0.00000 | 0.00097 | −0.64 | 0.64 | −1.56 | 14 |
| | KIAA0141 | | | | | | | |
| 1382939_at | translocated promoter region | −5.18 | 0.00005 | 0.00581 | −1.13 | 0.46 | −2.19 | 14 |
| | (predicted) | | | | | | | |
| 1382957_at | similar to cisplatin resistance- | −8.04 | 0.00000 | 0.00016 | −1.08 | 0.47 | −2.11 | 14 |
| | associated overexpressed | | | | | | | |
| | protein (predicted) | | | | | | | |
| 1382960_at | Transcribed locus | −5.95 | 0.00001 | 0.00208 | −0.77 | 0.59 | −1.70 | 14 |
| 1382972_at | Transcribed locus, strongly | 5.17 | 0.00005 | 0.00595 | 0.37 | 1.29 | 1.29 | 14 |
| | similar to XP_226713.2 | | | | | | | |
| | PREDICTED: similar to Src- | | | | | | | |
| | associated protein SAW | | | | | | | |
| | [Rattus norvegicus] | | | | | | | |
| 1383008_at | SMC4 structural maintenance | −5.19 | 0.00005 | 0.00581 | −0.98 | 0.51 | −1.97 | 14 |
| | of chromosomes 4-like 1 | | | | | | | |
| | (yeast) (predicted) | | | | | | | |
| 1383040_a_at | | −5.46 | 0.00003 | 0.00419 | −0.47 | 0.72 | −1.38 | 14 |
| 1383052_a_at | | −6.54 | 0.00000 | 0.00097 | −0.62 | 0.65 | −1.53 | 14 |
| 1383054_at | | −7.86 | 0.00000 | 0.00019 | −0.76 | 0.59 | −1.70 | 14 |
| 1383060_at | G kinase anchoring protein 1 | −5.82 | 0.00001 | 0.00255 | −0.44 | 0.74 | −1.35 | 14 |
| | (predicted) | | | | | | | |
| 1383085_at | Similar to Sh3bgrl protein | −5.20 | 0.00005 | 0.00576 | −0.86 | 0.55 | −1.81 | 14 |
| 1383179_at | Similar to hypothetical protein | −5.10 | 0.00006 | 0.00660 | −0.75 | 0.60 | −1.68 | 14 |
| | HSPC129 (predicted) | | | | | | | |
| 1383184_at | zinc and ring finger 1 | 5.03 | 0.00007 | 0.00731 | 0.39 | 1.31 | 1.31 | 14 |
| | (predicted) | | | | | | | |
| 1383334_at | Transcribed locus | −5.37 | 0.00003 | 0.00461 | −0.46 | 0.73 | −1.37 | 14 |
| 1383455_at | glutamyl-prolyl-tRNA | −6.80 | 0.00000 | 0.00072 | −0.72 | 0.61 | −1.65 | 14 |
| | synthetase (predicted) | | | | | | | |
| 1383535_at | ankyrin repeat and SOCS box- | 6.24 | 0.00001 | 0.00152 | 0.38 | 1.30 | 1.30 | 14 |
| | containing protein 8 (predicted) | | | | | | | |
| 1383615_a_at | similar to HECT domain | −6.06 | 0.00001 | 0.00178 | −1.08 | 0.47 | −2.11 | 14 |
| | containing 1 | | | | | | | |
| 1383687_at | | −5.40 | 0.00003 | 0.00441 | −0.43 | 0.74 | −1.34 | 14 |
| 1383776_at | Transcribed locus | 6.41 | 0.00000 | 0.00120 | 0.62 | 1.54 | 1.54 | 14 |
| 1383786_at | Transcribed locus | −5.13 | 0.00006 | 0.00628 | −0.50 | 0.71 | −1.41 | 14 |
| 1383825_at | radixin | −9.20 | 0.00000 | 0.00005 | −1.01 | 0.50 | −2.01 | 14 |
| 1383827_at | tousled-like kinase 1 | −6.09 | 0.00001 | 0.00178 | −1.25 | 0.42 | −2.37 | 14 |
| | (predicted) | | | | | | | |
| 1384125_at | myeloid/lymphoid or mixed- | −5.97 | 0.00001 | 0.00202 | −0.51 | 0.70 | −1.42 | 14 |
| | lineage leukemia 5 (trithorax | | | | | | | |
| | homolog, Drosophila) | | | | | | | |
| | (predicted) | | | | | | | |
| 1384131_at | ADP-ribosylation factor-like 6 | 6.10 | 0.00001 | 0.00176 | 0.70 | 1.63 | 1.63 | 14 |
| | interacting protein 2 (predicted) | | | | | | | |
| 1384146_at | Similar to CD69 antigen (p60, | −5.22 | 0.00005 | 0.00568 | −1.35 | 0.39 | −2.55 | 14 |
| | early T-cell activation antigen) | | | | | | | |
| 1384154_at | WW domain binding protein 4 | −5.33 | 0.00004 | 0.00483 | −0.52 | 0.70 | −1.43 | 14 |
| 1384260_at | Transcribed locus | −6.36 | 0.00000 | 0.00126 | −0.66 | 0.63 | −1.58 | 14 |
| 1384263_at | ATP-binding cassette, sub- | −7.27 | 0.00000 | 0.00040 | −0.72 | 0.61 | −1.64 | 14 |
| | family A (ABC1), member 13 | | | | | | | |
| | (predicted) | | | | | | | |
| | similar to hypothetical protein | | | | | | | |
| | MGC33214 (predicted) | | | | | | | |
| 1384339_s_at | casein kinase II, alpha 1 | −8.81 | 0.00000 | 0.00006 | −1.83 | 0.28 | −3.56 | 14 |
| | polypeptide | | | | | | | |
| 1384376_at | similar to FLJ14281 protein | −5.42 | 0.00003 | 0.00436 | −0.65 | 0.64 | −1.57 | 14 |
| 1384394_at | | −7.21 | 0.00000 | 0.00042 | −0.61 | 0.65 | −1.53 | 14 |
| 1384609_a_at | similar to RIKEN cDNA | −6.61 | 0.00000 | 0.00090 | −0.91 | 0.53 | −1.87 | 14 |
| | B230380D07 (predicted) | | | | | | | |
| 1384766_a_at | similar to PHD finger protein | −5.18 | 0.00005 | 0.00581 | −0.70 | 0.61 | −1.63 | 14 |
| | 14 isoform 1 | | | | | | | |
| 1384791_at | UDP-GlcNAc:betaGal beta- | −5.08 | 0.00006 | 0.00671 | −0.75 | 0.60 | −1.68 | 14 |
| | 1,3-N- | | | | | | | |
| | acetylglucosaminyltransferase | | | | | | | |
| | 1 (predicted) | | | | | | | |
| 1384792_at | formin binding protein 3 | −6.70 | 0.00000 | 0.00082 | −0.97 | 0.51 | −1.96 | 14 |
| | (predicted) | | | | | | | |
| 1384857_at | A kinase (PRKA) anchor | −5.62 | 0.00002 | 0.00338 | −1.05 | 0.48 | −2.07 | 14 |
| | protein (yotiao) 9 | | | | | | | |
| 1385006_at | alpha thalassemia/mental | −4.94 | 0.00009 | 0.00829 | −0.45 | 0.73 | −1.37 | 14 |
| | retardation syndrome X-linked | | | | | | | |
| | homolog (human) | | | | | | | |
| 1385038_at | similar to hedgehog-interacting | −9.94 | 0.00000 | 0.00003 | −0.80 | 0.57 | −1.75 | 14 |
| | protein | | | | | | | |
| 1385076_at | | −5.78 | 0.00001 | 0.00271 | −0.57 | 0.68 | −1.48 | 14 |
| 1385077_at | similar to golgi-specific | −7.83 | 0.00000 | 0.00019 | −1.05 | 0.48 | −2.07 | 14 |
| | brefeldin A-resistance guanine | | | | | | | |
| | nucleotide exchange factor 1 | | | | | | | |
| | (predicted) | | | | | | | |
| 1385101_a_at | Unknown (protein for | −5.53 | 0.00002 | 0.00383 | −0.97 | 0.51 | −1.96 | 14 |
| | MGC: 73017) | | | | | | | |
| 1385108_at | Transcribed locus | −4.83 | 0.00011 | 0.00946 | −1.27 | 0.42 | −2.40 | 14 |
| 1385240_at | WD repeat domain 33 | −4.81 | 0.00012 | 0.00958 | −0.93 | 0.52 | −1.91 | 14 |
| | (predicted) | | | | | | | |
| 1385320_at | similar to Pdz-containing | −5.26 | 0.00004 | 0.00530 | −0.47 | 0.72 | −1.38 | 14 |
| | protein | | | | | | | |
| 1385407_at | TCDD-inducible poly(ADP- | −5.46 | 0.00003 | 0.00417 | −1.34 | 0.39 | −2.54 | 14 |
| | ribose) polymerase (predicted) | | | | | | | |
| 1385408_at | similar to mKIAA0518 protein | −5.86 | 0.00001 | 0.00236 | −1.31 | 0.40 | −2.49 | 14 |
| 1385689_at | Transcribed locus | −4.83 | 0.00011 | 0.00948 | −0.68 | 0.62 | −1.60 | 14 |
| 1385852_at | CREB binding protein | −4.85 | 0.00011 | 0.00927 | −0.53 | 0.69 | −1.44 | 14 |
| | hypothetical gene supported by | | | | | | | |
| | NM_133381 | | | | | | | |
| 1385931_at | hook homolog 3 | −7.30 | 0.00000 | 0.00040 | −1.66 | 0.32 | −3.16 | 14 |
| 1385999_at | YME1-like 1 (S. cerevisiae) | −4.80 | 0.00012 | 0.00964 | −0.63 | 0.64 | −1.55 | 14 |
| 1386191_a_at | Transcribed locus | 5.22 | 0.00005 | 0.00568 | 0.46 | 1.37 | 1.37 | 14 |
| 1386641_at | Transcribed locus | −5.41 | 0.00003 | 0.00440 | −0.97 | 0.51 | −1.95 | 14 |
| 1386793_at | similar to zinc finger protein 61 | −5.28 | 0.00004 | 0.00514 | −0.59 | 0.66 | −1.51 | 14 |
| 1387087_at | CCAAT/enhancer binding | −5.43 | 0.00003 | 0.00435 | −0.61 | 0.66 | −1.52 | 14 |
| | protein (C/EBP), beta | | | | | | | |
| 1387306_a_at | early growth response 2 | 4.82 | 0.00012 | 0.00950 | 0.33 | 1.26 | 1.26 | 14 |
| 1387365_at | nuclear receptor subfamily 1, | −4.86 | 0.00011 | 0.00913 | −0.35 | 0.78 | −1.27 | 14 |
| | group H, member 3 | | | | | | | |
| 1387415_a_at | syntaxin binding protein 5 | 4.86 | 0.00011 | 0.00913 | 0.40 | 1.32 | 1.32 | 14 |
| | (tomosyn) | | | | | | | |
| 1387458_at | ring finger protein 4 | 7.05 | 0.00000 | 0.00051 | 0.75 | 1.69 | 1.69 | 14 |
| 1387664_at | ATPase, H+ transporting, V1 | 5.55 | 0.00002 | 0.00375 | 0.46 | 1.38 | 1.38 | 14 |
| | subunit B, isoform 2 | | | | | | | |
| 1387757_at | liver regeneration p-53 related | 5.19 | 0.00005 | 0.00581 | 0.51 | 1.42 | 1.42 | 14 |
| | protein | | | | | | | |
| 1387760_a_at | one cut domain, family | −6.12 | 0.00001 | 0.00171 | −1.58 | 0.34 | −2.98 | 14 |
| | member 1 | | | | | | | |
| 1387789_at | v-ets erythroblastosis virus E26 | −6.61 | 0.00000 | 0.00090 | −0.58 | 0.67 | −1.50 | 14 |
| | oncogene like (avian) | | | | | | | |
| 1387915_at | Ratsg2 | −4.79 | 0.00013 | 0.00976 | −0.33 | 0.79 | −1.26 | 14 |
| 1387947_at | v-maf musculoaponeurotic | −5.08 | 0.00007 | 0.00674 | −0.80 | 0.57 | −1.74 | 14 |
| | fibrosarcoma oncogene family, | | | | | | | |
| | protein B (avian) | | | | | | | |
| 1388022_a_at | dynamin 1-like | 4.95 | 0.00009 | 0.00811 | 0.43 | 1.35 | 1.35 | 14 |
| 1388059_a_at | solute carrier family 11 | 5.75 | 0.00001 | 0.00280 | 0.43 | 1.35 | 1.35 | 14 |
| | (proton-coupled divalent metal | | | | | | | |
| | ion transporters), member 2 | | | | | | | |
| 1388089_a_at | ring finger protein 4 | 5.73 | 0.00002 | 0.00289 | 0.50 | 1.41 | 1.41 | 14 |
| 1388157_at | myristoylated alanine rich | −5.76 | 0.00001 | 0.00276 | −0.51 | 0.70 | −1.43 | 14 |
| | protein kinase C substrate | | | | | | | |
| 1388196_at | NCK-associated protein 1 | 5.31 | 0.00004 | 0.00500 | 0.48 | 1.40 | 1.40 | 14 |
| 1388251_at | protein kinase C, lambda | 5.21 | 0.00005 | 0.00571 | 0.55 | 1.47 | 1.47 | 14 |
| 1388313_at | ribosomal protein s25 | −4.83 | 0.00011 | 0.00946 | −0.63 | 0.65 | −1.54 | 14 |
| 1388353_at | proliferation-associated 2G4, | −6.35 | 0.00000 | 0.00127 | −0.67 | 0.63 | −1.59 | 14 |
| | 38 kDa | | | | | | | |
| 1388388_at | Protein phosphatase 2, | −5.32 | 0.00004 | 0.00487 | −0.41 | 0.75 | −1.33 | 14 |
| | regulatory subunit B (B56), | | | | | | | |
| | delta isoform (predicted) | | | | | | | |
| 1388396_at | serine/threonine kinase 25 | −5.49 | 0.00003 | 0.00404 | −0.34 | 0.79 | −1.27 | 14 |
| | (STE20 homolog, yeast) | | | | | | | |
| 1388503_at | similar to CREBBP/EP300 | −6.06 | 0.00001 | 0.00178 | −0.40 | 0.76 | −1.32 | 14 |
| | inhibitory protein 1 | | | | | | | |
| 1388714_at | elongation factor RNA | −5.88 | 0.00001 | 0.00229 | −0.46 | 0.73 | −1.37 | 14 |
| | polymerase II (predicted) | | | | | | | |
| 1388735_at | Similar to keratin associated | 4.84 | 0.00011 | 0.00945 | 0.50 | 1.41 | 1.41 | 14 |
| | protein 10-6 | | | | | | | |
| 1388752_at | BCL2-associated transcription | −4.81 | 0.00012 | 0.00958 | −0.40 | 0.76 | −1.32 | 14 |
| | factor 1 (predicted) | | | | | | | |
| 1388849_at | Protease, serine, 25 (predicted) | −5.97 | 0.00001 | 0.00202 | −0.50 | 0.71 | −1.41 | 14 |
| 1388888_at | Transcribed locus | 5.13 | 0.00006 | 0.00632 | 0.40 | 1.32 | 1.32 | 14 |
| 1389268_at | similar to DNA polymerase | −5.21 | 0.00005 | 0.00571 | −0.37 | 0.77 | −1.29 | 14 |
| | lambda | | | | | | | |
| 1389307_at | similar to Amyloid beta (A4) | −4.91 | 0.00010 | 0.00854 | −0.49 | 0.71 | −1.40 | 14 |
| | precursor-like protein 1 | | | | | | | |
| 1389419_at | Transcribed locus | −6.54 | 0.00000 | 0.00097 | −1.30 | 0.40 | −2.47 | 14 |
| 1389432_at | pre-B-cell leukemia | −4.93 | 0.00009 | 0.00829 | −0.55 | 0.69 | −1.46 | 14 |
| | transcription factor 2 | | | | | | | |
| 1389444_at | Transcribed locus | −6.84 | 0.00000 | 0.00068 | −1.06 | 0.48 | −2.09 | 14 |
| 1389806_at | Transcribed locus | −7.63 | 0.00000 | 0.00026 | −0.54 | 0.69 | −1.46 | 14 |
| 1389868_at | similar to RCK | −6.34 | 0.00000 | 0.00128 | −1.47 | 0.36 | −2.76 | 14 |
| 1389963_at | P55 mRNA for p55 protein | −5.54 | 0.00002 | 0.00378 | −0.44 | 0.74 | −1.35 | 14 |
| 1389986_at | LOC499304 | −5.39 | 0.00003 | 0.00449 | −2.57 | 0.17 | −5.93 | 14 |
| 1389989_at | alpha thalassemia/mental | −4.93 | 0.00009 | 0.00829 | −0.54 | 0.69 | −1.45 | 14 |
| | retardation syndrome X-linked | | | | | | | |
| | homolog (human) | | | | | | | |
| 1389998_at | Nuclear receptor subfamily 2, | −6.03 | 0.00001 | 0.00187 | −0.69 | 0.62 | −1.61 | 14 |
| | group F, member 2 | | | | | | | |
| 1390048_at | serine/arginine repetitive | −5.81 | 0.00001 | 0.00256 | −1.04 | 0.49 | −2.05 | 14 |
| | matrix 2 (predicted) | | | | | | | |
| 1390120_a_at | ring finger protein 1 | −5.70 | 0.00002 | 0.00299 | −0.36 | 0.78 | −1.28 | 14 |
| 1390121_at | GLIS family zinc finger 2 | 4.81 | 0.00012 | 0.00958 | 0.43 | 1.34 | 1.34 | 14 |
| | (predicted) | | | | | | | |
| 1390227_at | CDNA clone IMAGE: 7300848 | −5.91 | 0.00001 | 0.00219 | −1.03 | 0.49 | −2.04 | 14 |
| 1390360_a_at | similar to Safb2 protein | −4.79 | 0.00013 | 0.00976 | −0.48 | 0.72 | −1.39 | 14 |
| 1390410_at | Transcribed locus | −4.79 | 0.00012 | 0.00973 | −0.49 | 0.71 | −1.40 | 14 |
| 1390436_at | Autophagy 7-like (S. cerevisiae) | −7.88 | 0.00000 | 0.00019 | −1.46 | 0.36 | −2.76 | 14 |
| | (predicted) | | | | | | | |
| 1390448_at | similar to 1110065L07Rik | 5.08 | 0.00007 | 0.00674 | 0.32 | 1.25 | 1.25 | 14 |
| | protein (predicted) | | | | | | | |
| 1390454_at | 4-nitrophenylphosphatase | −5.47 | 0.00003 | 0.00415 | −0.41 | 0.75 | −1.33 | 14 |
| | domain and non-neuronal | | | | | | | |
| | SNAP25-like protein homolog | | | | | | | |
| | 1 (C. elegans) (predicted) | | | | | | | |
| 1390576_at | Transcribed locus | −5.10 | 0.00006 | 0.00660 | −0.67 | 0.63 | −1.59 | 14 |
| 1390660_at | T-box 2 (predicted) | 5.01 | 0.00008 | 0.00752 | 0.40 | 1.32 | 1.32 | 14 |
| 1390706_at | spectrin beta 2 | −5.55 | 0.00002 | 0.00376 | −0.71 | 0.61 | −1.64 | 14 |
| 1390739_at | similar to zinc finger protein | −5.51 | 0.00003 | 0.00395 | −0.52 | 0.70 | −1.43 | 14 |
| | 609 | | | | | | | |
| | similar to zinc finger protein | | | | | | | |
| | 609 | | | | | | | |
| 1390777_at | sterol-C5-desaturase (fungal | −6.59 | 0.00000 | 0.00090 | −0.70 | 0.61 | −1.63 | 14 |
| | ERG3, delta-5-desaturase) | | | | | | | |
| | homolog (S. cerevisae) | | | | | | | |
| 1390779_at | Similar to phosphoseryl-tRNA | −4.86 | 0.00011 | 0.00913 | −0.64 | 0.64 | −1.56 | 14 |
| | kinase | | | | | | | |
| 1390813_at | Similar to RNA-binding | −5.19 | 0.00005 | 0.00581 | −0.62 | 0.65 | −1.54 | 14 |
| | protein Musashi2-S | | | | | | | |
| 1390884_a_at | UDP-GlcNAc:betaGal beta- | 4.87 | 0.00010 | 0.00904 | 0.49 | 1.40 | 1.40 | 14 |
| | 1,3-N- | | | | | | | |
| | acetylglucosaminyltransferase | | | | | | | |
| | 7 (predicted) | | | | | | | |
| 1391021_at | similar to KIAA1749 protein | −7.55 | 0.00000 | 0.00027 | −0.74 | 0.60 | −1.67 | 14 |
| | (predicted) | | | | | | | |
| 1391170_at | similar to mKIAA1757 protein | −9.21 | 0.00000 | 0.00005 | −2.01 | 0.25 | −4.04 | 14 |
| | (predicted) | | | | | | | |
| 1391222_at | similar to Nedd4 binding | −5.91 | 0.00001 | 0.00219 | −0.81 | 0.57 | −1.76 | 14 |
| | protein 1 (predicted) | | | | | | | |
| 1391297_at | REST corepressor 1 (predicted) | −5.17 | 0.00005 | 0.00595 | −0.92 | 0.53 | −1.89 | 14 |
| 1391578_at | | −8.48 | 0.00000 | 0.00009 | −1.11 | 0.46 | −2.15 | 14 |
| 1391584_at | Transcribed locus | 6.04 | 0.00001 | 0.00185 | 0.45 | 1.37 | 1.37 | 14 |
| 1391625_at | Wiskott-Aldrich syndrome-like | −10.52 | 0.00000 | 0.00002 | −1.28 | 0.41 | −2.43 | 14 |
| | (human) | | | | | | | |
| 1391669_at | protein tyrosine phosphatase, | −6.21 | 0.00001 | 0.00156 | −0.82 | 0.56 | −1.77 | 14 |
| | receptor type, B (predicted) | | | | | | | |
| 1391689_at | similar to Retinoblastoma- | −9.06 | 0.00000 | 0.00005 | −1.20 | 0.44 | −2.30 | 14 |
| | binding protein 2 (RBBP-2) | | | | | | | |
| 1391701_at | MYST histone | −5.18 | 0.00005 | 0.00581 | −0.98 | 0.51 | −1.97 | 14 |
| | acetyltransferase (monocytic | | | | | | | |
| | leukemia) 3 (predicted) | | | | | | | |
| 1391743_at | ELAV (embryonic lethal, | −4.80 | 0.00012 | 0.00964 | −1.36 | 0.39 | −2.58 | 14 |
| | abnormal vision, Drosophila)- | | | | | | | |
| | like 1 (Hu antigen R) | | | | | | | |
| | (predicted) | | | | | | | |
| 1391830_at | copine VIII (predicted) | −5.22 | 0.00005 | 0.00568 | −1.08 | 0.47 | −2.12 | 14 |
| 1391838_at | ankyrin repeat domain 11 | −7.81 | 0.00000 | 0.00019 | −1.15 | 0.45 | −2.23 | 14 |
| | (predicted) | | | | | | | |
| 1391848_at | RNA binding motif protein 27 | −7.02 | 0.00000 | 0.00053 | −0.76 | 0.59 | −1.70 | 14 |
| | (predicted) | | | | | | | |
| 1391968_at | Similar to expressed sequence | −4.89 | 0.00010 | 0.00883 | −0.69 | 0.62 | −1.62 | 14 |
| | AA415817 | | | | | | | |
| 1392000_at | Similar to PHD finger protein | 5.01 | 0.00008 | 0.00742 | 0.45 | 1.37 | 1.37 | 14 |
| | 14 isoform 1 | | | | | | | |
| 1392061_at | minichromosome maintenance | 5.34 | 0.00004 | 0.00482 | 0.54 | 1.46 | 1.46 | 14 |
| | deficient 10 (S. cerevisiae) | | | | | | | |
| | (predicted) | | | | | | | |
| 1392269_at | transcriptional regulator, | −6.23 | 0.00001 | 0.00153 | −1.13 | 0.46 | −2.19 | 14 |
| | SIN3A (yeast) (predicted) | | | | | | | |
| 1392277_at | | −7.29 | 0.00000 | 0.00040 | −0.48 | 0.72 | −1.40 | 14 |
| 1392322_at | GTPase, IMAP family member 7 | −4.83 | 0.00012 | 0.00948 | −0.29 | 0.82 | −1.22 | 14 |
| 1392472_at | similar to myocyte enhancer | −9.77 | 0.00000 | 0.00003 | −0.88 | 0.54 | −1.84 | 14 |
| | factor 2C | | | | | | | |
| 1392552_at | similar to transcription | −6.15 | 0.00001 | 0.00169 | −0.96 | 0.51 | −1.95 | 14 |
| | repressor p66 (predicted) | | | | | | | |
| 1392564_at | myeloid/lymphoid or mixed- | −6.13 | 0.00001 | 0.00171 | −0.57 | 0.68 | −1.48 | 14 |
| | lineage leukemia 5 (trithorax | | | | | | | |
| | homolog, Drosophila) | | | | | | | |
| | (predicted) | | | | | | | |
| 1392629_a_at | similar to MADP-1 protein | −4.93 | 0.00009 | 0.00829 | −0.82 | 0.57 | −1.77 | 14 |
| | (predicted) | | | | | | | |
| 1392738_at | similar to KIAA1096 protein | −5.88 | 0.00001 | 0.00231 | −0.75 | 0.59 | −1.68 | 14 |
| 1392825_at | LOC499256 | −5.20 | 0.00005 | 0.00580 | −0.93 | 0.53 | −1.90 | 14 |
| 1392864_at | Rho GTPase activating protein | −8.05 | 0.00000 | 0.00016 | −1.37 | 0.39 | −2.58 | 14 |
| | 5 (predicted) | | | | | | | |
| 1392932_at | leukocyte receptor cluster | −4.81 | 0.00012 | 0.00958 | −0.79 | 0.58 | −1.73 | 14 |
| | (LRC) member 8 (predicted) | | | | | | | |
| 1392936_at | similar to RNA binding motif | −4.82 | 0.00012 | 0.00950 | −0.88 | 0.54 | −1.85 | 14 |
| | protein 25 | | | | | | | |
| 1392984_at | copine III (predicted) | −7.83 | 0.00000 | 0.00019 | −0.95 | 0.52 | −1.93 | 14 |
| 1393151_at | | 5.03 | 0.00007 | 0.00726 | 0.65 | 1.57 | 1.57 | 14 |
| 1393226_at | Transcribed locus | −4.94 | 0.00009 | 0.00828 | −0.73 | 0.60 | −1.66 | 14 |
| 1393290_at | similar to myocyte enhancer | −5.65 | 0.00002 | 0.00327 | −0.50 | 0.71 | −1.42 | 14 |
| | factor 2C | | | | | | | |
| 1393322_at | TAF15 RNA polymerase II, | −6.18 | 0.00001 | 0.00162 | −1.00 | 0.50 | −2.00 | 14 |
| | TATA box binding protein | | | | | | | |
| | (TBP)-associated factor | | | | | | | |
| | (predicted) | | | | | | | |
| 1393378_at | | −5.72 | 0.00002 | 0.00293 | −0.52 | 0.70 | −1.43 | 14 |
| 1393443_a_at | similar to CGI-112 protein | −5.33 | 0.00004 | 0.00483 | −0.47 | 0.72 | −1.39 | 14 |
| | (predicted) | | | | | | | |
| 1393505_x_at | similar to RIKEN cDNA | −7.60 | 0.00000 | 0.00026 | −0.69 | 0.62 | −1.61 | 14 |
| | B230380D07 (predicted) | | | | | | | |
| 1393511_at | similar to galactose-3-O- | 5.10 | 0.00006 | 0.00655 | 0.41 | 1.33 | 1.33 | 14 |
| | sulfotransferase 4 | | | | | | | |
| 1393560_at | | −4.91 | 0.00010 | 0.00852 | −0.51 | 0.70 | −1.42 | 14 |
| 1393576_at | Transcribed locus | −4.82 | 0.00012 | 0.00950 | −0.62 | 0.65 | −1.54 | 14 |
| 1393593_at | similar to KIAA0597 protein | 5.43 | 0.00003 | 0.00435 | 0.57 | 1.48 | 1.48 | 14 |
| 1393639_at | myosin X (predicted) | −4.95 | 0.00009 | 0.00811 | −0.59 | 0.67 | −1.50 | 14 |
| 1393790_at | HRAS-like suppressor | 5.44 | 0.00003 | 0.00432 | 0.44 | 1.35 | 1.35 | 14 |
| | (predicted) | | | | | | | |
| 1393798_at | alpha thalassemia/mental | −5.00 | 0.00008 | 0.00757 | −0.84 | 0.56 | −1.79 | 14 |
| | retardation syndrome X-linked | | | | | | | |
| | homolog (human) | | | | | | | |
| 1393804_at | similar to hypothetical protein | −6.79 | 0.00000 | 0.00073 | −0.85 | 0.56 | −1.80 | 14 |
| | FLJ22490 (predicted) | | | | | | | |
| 1393809_at | Tnf receptor-associated factor 6 | −8.48 | 0.00000 | 0.00009 | −0.90 | 0.53 | −1.87 | 14 |
| | (predicted) | | | | | | | |
| 1393811_at | similar to putative repair and | −6.08 | 0.00001 | 0.00178 | −0.79 | 0.58 | −1.73 | 14 |
| | recombination helicase | | | | | | | |
| | RAD26L | | | | | | | |
| 1393910_at | similar to Fam13a1 protein | −4.85 | 0.00011 | 0.00921 | −0.81 | 0.57 | −1.75 | 14 |
| | (predicted) | | | | | | | |
| 1393981_at | similar to KIAA0423 | −5.24 | 0.00005 | 0.00556 | −0.57 | 0.68 | −1.48 | 14 |
| | (predicted) | | | | | | | |
| 1394003_at | similar to DNA polymerase | −5.59 | 0.00002 | 0.00349 | −0.59 | 0.67 | −1.50 | 14 |
| | epsilon p17 subunit (DNA | | | | | | | |
| | polymerase epsilon subunit 3) | | | | | | | |
| | (Chromatin accessibility | | | | | | | |
| | complex 17) (HuCHRAC17) | | | | | | | |
| | (CHRAC-17) | | | | | | | |
| 1394220_at | Similar to hypothetical protein | 5.46 | 0.00003 | 0.00417 | 0.43 | 1.34 | 1.34 | 14 |
| | (predicted) | | | | | | | |
| 1394243_at | similar to spermine synthase | −6.11 | 0.00001 | 0.00175 | −0.60 | 0.66 | −1.51 | 14 |
| 1394436_at | sperm associated antigen 9 | −6.60 | 0.00000 | 0.00090 | −0.91 | 0.53 | −1.88 | 14 |
| | (predicted) | | | | | | | |
| 1394497_at | similar to TCF7L2 protein | −8.03 | 0.00000 | 0.00016 | −1.06 | 0.48 | −2.08 | 14 |
| 1394594_at | Transcribed locus | 5.09 | 0.00006 | 0.00671 | 0.42 | 1.34 | 1.34 | 14 |
| 1394715_at | Dicer1, Dcr-1 homolog | 5.14 | 0.00006 | 0.00627 | 0.54 | 1.46 | 1.46 | 14 |
| | (Drosophila) (predicted) | | | | | | | |
| 1394740_at | | 5.41 | 0.00003 | 0.00440 | 0.52 | 1.43 | 1.43 | 14 |
| 1394742_at | Transcribed locus | −5.73 | 0.00002 | 0.00289 | −0.98 | 0.51 | −1.98 | 14 |
| 1394746_at | hect (homologous to the E6-AP | −7.32 | 0.00000 | 0.00039 | −0.94 | 0.52 | −1.91 | 14 |
| | (UBE3A) carboxyl terminus) | | | | | | | |
| | domain and RCC1 (CHC1)-like | | | | | | | |
| | domain (RLD) 1 (predicted) | | | | | | | |
| 1394814_at | translocated promoter region | −6.13 | 0.00001 | 0.00171 | −0.63 | 0.64 | −1.55 | 14 |
| | (predicted) | | | | | | | |
| 1394849_at | Transcribed locus | −5.22 | 0.00005 | 0.00569 | −1.61 | 0.33 | −3.05 | 14 |
| 1394865_at | Transmembrane protein 7 | −7.85 | 0.00000 | 0.00019 | −0.92 | 0.53 | −1.90 | 14 |
| | (predicted) | | | | | | | |
| 1394965_at | enthoprotin | 5.30 | 0.00004 | 0.00503 | 0.40 | 1.32 | 1.32 | 14 |
| 1394969_at | Transcribed locus | 5.40 | 0.00003 | 0.00441 | 0.39 | 1.31 | 1.31 | 14 |
| 1394985_at | early endosome antigen 1 | −7.60 | 0.00000 | 0.00026 | −1.00 | 0.50 | −2.00 | 14 |
| | (predicted) | | | | | | | |
| 1395211_s_at | supervillin (predicted) | −8.74 | 0.00000 | 0.00007 | −0.98 | 0.51 | −1.97 | 14 |
| 1395237_at | eukaryotic translation initiation | −8.31 | 0.00000 | 0.00012 | −0.87 | 0.55 | −1.83 | 14 |
| | factor 5B | | | | | | | |
| 1395264_at | similar to Rap1-interacting | −6.85 | 0.00000 | 0.00067 | −0.95 | 0.52 | −1.93 | 14 |
| | factor 1 | | | | | | | |
| 1395331_at | similar to hypothetical protein | 4.84 | 0.00011 | 0.00945 | 0.31 | 1.24 | 1.24 | 14 |
| | CL25084 (predicted) | | | | | | | |
| 1395338_at | leucine-rich PPR-motif | 5.24 | 0.00005 | 0.00555 | 0.75 | 1.68 | 1.68 | 14 |
| | containing (predicted) | | | | | | | |
| 1395516_at | similar to hypothetical protein | −4.89 | 0.00010 | 0.00883 | −0.59 | 0.66 | −1.51 | 14 |
| | FLJ10154 (predicted) | | | | | | | |
| 1395565_at | COP9 signalosome subunit 4 | 5.55 | 0.00002 | 0.00376 | 0.40 | 1.32 | 1.32 | 14 |
| 1395610_at | similar to Hypothetical protein | 5.66 | 0.00002 | 0.00325 | 0.33 | 1.26 | 1.26 | 14 |
| | MGC30714 | | | | | | | |
| 1395616_at | similar to Ab2-008 (predicted) | −5.03 | 0.00007 | 0.00729 | −0.50 | 0.71 | −1.42 | 14 |
| 1395625_at | Transcribed locus | −6.03 | 0.00001 | 0.00187 | −0.76 | 0.59 | −1.70 | 14 |
| 1395739_at | similar to RIKEN cDNA | 5.05 | 0.00007 | 0.00698 | 0.54 | 1.46 | 1.46 | 14 |
| | C920006C10 (predicted) | | | | | | | |
| 1395814_at | Transcribed locus | −5.09 | 0.00006 | 0.00663 | −0.78 | 0.58 | −1.71 | 14 |
| 1395976_at | similar to phosphoinositol 4- | −6.37 | 0.00000 | 0.00126 | −0.57 | 0.67 | −1.49 | 14 |
| | phosphate adaptor protein-2 | | | | | | | |
| 1395981_at | helicase, ATP binding 1 | −5.76 | 0.00001 | 0.00276 | −0.62 | 0.65 | −1.54 | 14 |
| | (predicted) | | | | | | | |
| 1396036_at | Ral GEF with PH domain and | −6.67 | 0.00000 | 0.00084 | −1.04 | 0.49 | −2.06 | 14 |
| | SH3 binding motif 2 | | | | | | | |
| | (predicted) | | | | | | | |
| 1396063_at | DEK oncogene (DNA binding) | −4.82 | 0.00012 | 0.00952 | −0.63 | 0.65 | −1.55 | 14 |
| 1396100_at | similar to RIKEN cDNA | −5.15 | 0.00006 | 0.00610 | −0.56 | 0.68 | −1.47 | 14 |
| | 2010009L17 (predicted) | | | | | | | |
| 1396170_at | WW domain binding protein 4 | −7.78 | 0.00000 | 0.00020 | −0.77 | 0.59 | −1.71 | 14 |
| 1396187_at | Hypothetical protein | 5.14 | 0.00006 | 0.00622 | 0.51 | 1.43 | 1.43 | 14 |
| | LOC606294 | | | | | | | |
| 1396202_at | Transcribed locus | 4.97 | 0.00008 | 0.00795 | 0.52 | 1.44 | 1.44 | 14 |
| 1396403_at | | −9.07 | 0.00000 | 0.00005 | −1.01 | 0.50 | −2.02 | 14 |
| 1396803_at | similar to THO complex 2 | −7.09 | 0.00000 | 0.00050 | −0.90 | 0.54 | −1.86 | 14 |
| 1397203_at | PRP4 pre-mRNA processing | −6.18 | 0.00001 | 0.00162 | −0.67 | 0.63 | −1.59 | 14 |
| | factor 4 homolog B (yeast) | | | | | | | |
| | (predicted) | | | | | | | |
| 1397234_at | G patch domain containing 1 | −5.65 | 0.00002 | 0.00326 | −0.49 | 0.71 | −1.40 | 14 |
| | (predicted) | | | | | | | |
| 1397367_at | A disintegrin and | 5.05 | 0.00007 | 0.00698 | 0.47 | 1.38 | 1.38 | 14 |
| | metalloprotease domain 23 | | | | | | | |
| | (predicted) | | | | | | | |
| 1397508_at | similar to RIKEN cDNA | −5.08 | 0.00006 | 0.00671 | −0.62 | 0.65 | −1.54 | 14 |
| | 2310005B10 | | | | | | | |
| 1397552_at | echinoderm microtubule | −8.47 | 0.00000 | 0.00009 | −1.39 | 0.38 | −2.62 | 14 |
| | associated protein like 4 | | | | | | | |
| | (predicted) | | | | | | | |
| 1397627_at | diaphanous homolog 1 | −5.07 | 0.00007 | 0.00680 | −0.52 | 0.70 | −1.43 | 14 |
| | (Drosophila) (predicted) | | | | | | | |
| 1397647_at | solute carrier family 25 | 5.51 | 0.00003 | 0.00395 | 0.62 | 1.54 | 1.54 | 14 |
| | (mitochondrial carrier; | | | | | | | |
| | ornithine transporter) member | | | | | | | |
| | 15 (predicted) | | | | | | | |
| 1397669_at | Chemokine (C—C motif) | 5.78 | 0.00001 | 0.00271 | 0.51 | 1.43 | 1.43 | 14 |
| | receptor 6 (predicted) | | | | | | | |
| 1397674_at | eukaryotic translation initiation | −6.44 | 0.00000 | 0.00116 | −0.76 | 0.59 | −1.69 | 14 |
| | factor 3, subunit 8, 110 kDa | | | | | | | |
| | (predicted) | | | | | | | |
| 1397676_at | Similar to osteoclast inhibitory | −6.68 | 0.00000 | 0.00084 | −1.34 | 0.39 | −2.54 | 14 |
| | lectin | | | | | | | |
| 1397758_at | Similar to choline | −4.83 | 0.00011 | 0.00946 | −0.38 | 0.77 | −1.30 | 14 |
| | phosphotransferase 1; | | | | | | | |
| | cholinephosphotransferase 1 | | | | | | | |
| | alpha; | | | | | | | |
| | cholinephosphotransferase 1 | | | | | | | |
| 1397959_at | similar to RIKEN cDNA | −6.39 | 0.00000 | 0.00123 | −1.14 | 0.45 | −2.20 | 14 |
| | D130059P03 gene (predicted) | | | | | | | |
| 1398311_a_at | kinase D-interacting substance | 5.14 | 0.00006 | 0.00627 | 0.44 | 1.36 | 1.36 | 14 |
| | 220 | | | | | | | |
| 1398351_at | Ubiquitin specific protease 7 | −5.60 | 0.00002 | 0.00349 | −0.42 | 0.75 | −1.34 | 14 |
| | (herpes virus-associated) | | | | | | | |
| | (predicted) | | | | | | | |
| 1398420_at | Similar to E3 ubiquitin ligase | −5.33 | 0.00004 | 0.00483 | −0.94 | 0.52 | −1.92 | 14 |
| | SMURF2 (predicted) | | | | | | | |
| 1398436_at | ubiquitin specific protease 42 | −6.36 | 0.00000 | 0.00126 | −0.76 | 0.59 | −1.69 | 14 |
| | (predicted) | | | | | | | |
| 1398486_at | CDNA clone MGC: 93990 | −8.09 | 0.00000 | 0.00016 | −1.53 | 0.35 | −2.89 | 14 |
| | IMAGE: 7115381 | | | | | | | |
| 1398522_at | similar to Ab2-034 (predicted) | −4.92 | 0.00009 | 0.00832 | −0.51 | 0.70 | −1.42 | 14 |
| 1398553_at | similar to CGI-100-like protein | −6.91 | 0.00000 | 0.00062 | −1.68 | 0.31 | −3.20 | 14 |
| 1398834_at | mitogen activated protein | −4.94 | 0.00009 | 0.00828 | −0.32 | 0.80 | −1.25 | 14 |
| | kinase kinase 2 | | | | | | | |
| 1398926_at | prefoldin 1 (predicted) | −5.95 | 0.00001 | 0.00208 | −0.48 | 0.72 | −1.40 | 14 |
| 1398963_at | TAF10 RNA polymerase II, | −5.42 | 0.00003 | 0.00436 | −0.41 | 0.75 | −1.33 | 14 |
| | TATA box binding protein | | | | | | | |
| | (TBP)-associated factor | | | | | | | |
| | (predicted) | | | | | | | |
| 1399099_at | heterogeneous nuclear | −4.94 | 0.00009 | 0.00829 | −0.54 | 0.69 | −1.46 | 14 |
| | ribonucleoprotein U-like 1 | | | | | | | |
| | (predicted) | | | | | | | |
| 1399140_at | Transcribed locus | −5.16 | 0.00005 | 0.00597 | −0.49 | 0.71 | −1.40 | 14 |
| AFFX-BioB- | Biotin synthase | −4.89 | 0.00010 | 0.00879 | −0.64 | 0.64 | −1.56 | 14 |
| M_at | biotin synthesis, sulfur | | | | | | | |
| | insertion? | | | | | | | |
| AFFX- | dethiobiotin synthetase | −4.92 | 0.00009 | 0.00834 | −0.70 | 0.62 | −1.62 | 14 |
| BioDn-5_at | | | | | | | | |
| AFFX-r2-Ec- | dethiobiotin synthetase | −5.41 | 0.00003 | 0.00440 | −0.51 | 0.70 | −1.43 | 14 |
| bioD-5_at | | | | | | | | |

### EXAMPLE 3

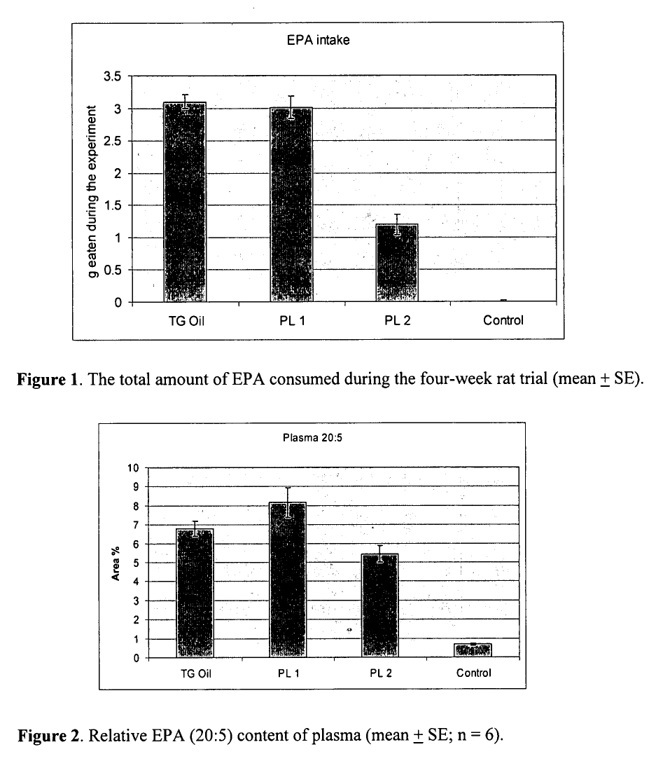

The effect of dietary supplementation of omega-3 phospholipids for 12 weeks on cognitive function, quality of life and behavioral outcome in children with ADHD have been investigated. Four children with ADHD were recruited, 2 children received a placebo (olive oil), 1 child received omega-3 phospholipids (700 mg/day, EPA:DHA=2:1) and the last child received omega-3 phospholipids (350 mg/day, EPA:DHA=2:1). Inclusion criteria were age (8-12 years), diagnosis of ADHD according to DSM-IV criteria, exhibiting symptoms of essential fatty acid deficiency and permission from parent/guardian. Exclusion criteria were use of dietary supplement containing omega-3 or omega-6 in the previous 6 months, consuming more than 2 fish meals per week, receiving medical treatment for a major health condition such as diabetes, depression or having a bleeding disorder. At baseline, after 4 weeks and after 12 weeks the child's cognitive ability was measured using computerized cognitive tests from Cogstate (Melbourne, Australia) as well as the conventional cognitive test from TEA-Ch [45] and WASI (Wechshler Abbreviated Scales of Intelligence) [46]. The parent/guardian completed a World Health Organization-Quality of life questionnaire [33], symptoms check list, BRIEF (Behavioral Rating Inventory of Executive Function) [47] and Conners' rating scale-revised (SCR-R) [48]. For each subject at each assessment, an average of the standardized scores was computed. The results are shown in FIG. 1, showing an improvement of the cognitive performance for the children receiving omega-3 phospholipids compared to baseline. None of the children receiving placebo completed the trial.

### EXAMPLE 4

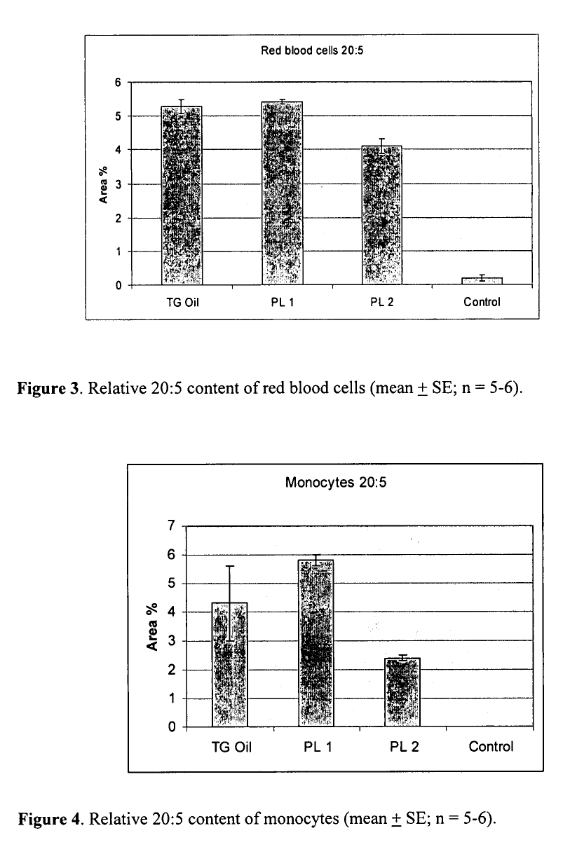

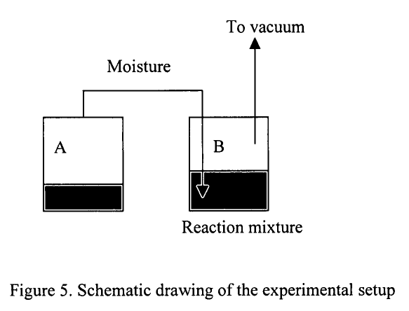

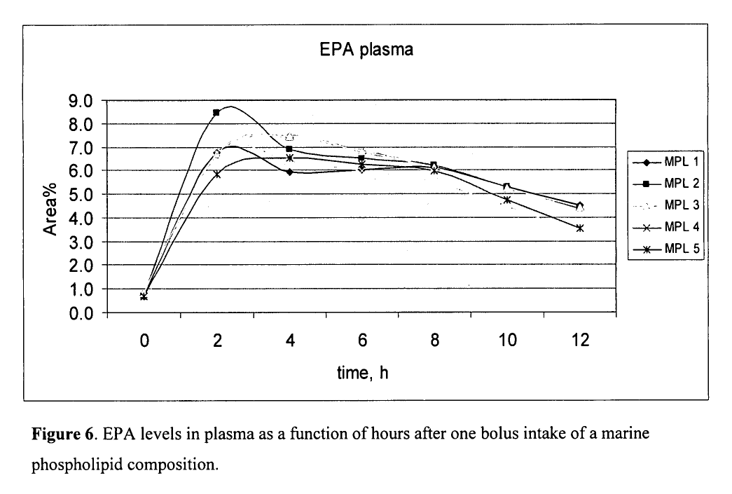

The effect of dietary supplementation of omega-3 phospholipids for 12 weeks on cognitive function, quality of life and behavioral outcome in healthy children was investigated. 20 children was recruited 10 children received placebo (olive oil) and 10 children received omega-3 phospholipids (700 mg omega-3/day, EPA:DHA=2:1). Inclusion criteria were male and female age 8-12 years and permission from parent/guardian. Exclusion criteria were use of omega-3 and omega-6 dietary supplement, consuming more than 2 meals of fish per week, receiving medical treatment for any major health conditions such as diabetes, history of traumatic brain injury, symptoms on ADHD according to DSM-IV criteria and bleeding disorders. At baseline, after 4 weeks and after 12 weeks the child's cognitive ability was measured using computerized cognitive tests from Cogstate (Melbourne, Australia) as well as the conventional cognitive test from TEA-Ch [45] and Weschler Abbreviated Scales of Intelligence (WASI) [46]. The parent/guardian completed a World Health Organization-Quality of life questionnaire [33], symptoms check list, BRIEF (Behavioral Rating Inventory of Executive Function) [47] and Conners' rating scale-revised (SCR-R) [48]. For each subject at each assessment, an average of the standardized scores was computed. The results are shown in FIG. 2, showing an improvement of the cognitive performance for the healthy children receiving omega-3 phospholipids compared to both placebo and baseline. One of the subjects was removed from the data set due to poor response. 3 children did not follow through the study after the initial baseline assessment.

### EXAMPLE 5

Fish oil (TG oil) were obtained from BLT (Aalesund, Norway) and EPA-rich phospholipids (PL 1) or DHA rich phospholipids (PL 2) were prepared according to the method disclosed in example 1. Next, the TAG and the phospholipids were mixed with the rat feed AIN-93 (without soybean oil). The concentration of EPA, DHA and 18:3 n-3 in the different diets can be seen in the table below (table 2).

TABLE 3
Amount of different fatty acid in the final feed products (g/100 g feed).

| | | g/100 g | g/100 g | g/100 g | SUM g/100 g |
|---|---|---|---|---|---|
| | | EPA | DHA | 18:3n3 | EPA + DHA + 18:3n3 |
| Control | T4 | 0 | 0 | 0.26 | 0.26 |
| TG Oil | T1 | 0.61 | 0.39 | 0.24 | 1.23 |
| PL 1 | T2 | 0.61 | 0.35 | 0.26 | 1.22 |
| PL 2 | T3 | 0.24 | 0.73 | 0.26 | 1.23 |

Thirty six newly weaned male Sprague Dawley rats (start weight 168±11 g) were used in the experiment. The rats were initially given low-essential oil rat feed, containing 20 g of sunflower oil and 10 g of flaxseed oil per kg of feed, for one week. After the first week, modified AIN-93 diet powder without the test oil was given to rats ad libitum until the start of the experiment. The experiment lasted for 30 days and the rats consumed the diets in table 3 ad libitum. However, before sampling feeding was stopped for 12 hours. Each rat was then individually anaesthetized with carbon dioxide, weighed and euthanized with cervical dislocation. The brain was then surgically removed from the rats and the fatty acid profiles of the total lipids (table 4), the phospholipids (table 5) and phospholipids sn-2 position (table 6) were determined using GC-FID and/or LC-MS.

TABLE 4
Fatty acid profile of the total lipids isolated from the rat brain (mmol/g lipids).

| | 18:1 | 18:2 | 18:3 n3 | 20:3 | ARA | EPA | 22:4 | 22:5 n6 | 22:5 n3 | DHA |
|---|---|---|---|---|---|---|---|---|---|---|
| TG-oil | 370 | 12 | 0.35 | 5.7 | 153.7 | 2.2 | 68.5 | 5.6 | 5.0 | 191.4 |
| PL-1 | 399 | 12 | 0.33 | 7.2 | 160.7 | 2.2 | 70.2 | 7.2 | 5.7 | 203.3 |
| PL-2 | 345 | 13 | 0.33 | 5.7 | 141.6 | 1.7 | 60.8 | 5.8 | 3.4 | 190.3 |
| Control | 363 | 12 | 0.30 | 5.2 | 174.9 | 0.1 | 81.4 | 7.6 | 1.7 | 189.6 |

TABLE 5
Fatty acid profile of the phospholipids isolated from the brain (mmol/g lipids).

| | 18:1 | 18:2 | 18:3 n3 | 20:3 | ARA | EPA | 22:4 | 22:5 n6 | 22:5 n3 | DHA |
|---|---|---|---|---|---|---|---|---|---|---|
| TG-oil | 260.8 | 9.9 | 0.2 | 4.5 | 147.17 | 1.9 | 72.8 | 5.6 | 5.3 | 196.8 |
| PL-1 | 289.9 | 10.9 | 0.1 | 4.2 | 115.07 | 1.4 | 54.7 | 4.3 | 4.5 | 170.1 |
| PL-2 | 232.6 | 2.1 | | 4.2 | 122.38 | 1.2 | 57.4 | 4.4 | 0.7 | 175.9 |
| Control | 288.8 | 4.4 | 0.6 | 3.5 | 181.60 | 0.1 | 86.8 | 7.3 | 0.5 | 213.1 |

TABLE 6
The fatty acid profile for the SN-2 position of the phospholipids
in the brain (mmol/g lipids).

| sn-2 | EPA | DHA | ARA | 18:2 | 22:5 | 20:3 | 22:4 | 18:1 |
|---|---|---|---|---|---|---|---|---|
| TG-oil | 1.6 | 164.7 | 123.6 | 9.8 | 5.0 | 3.9 | 72.8 | 197.4 |
| PL-1 | 1.3 | 146.3 | 96.5 | 11.6 | 3.7 | 4.1 | 51.8 | 260.2 |
| PL-2 | 1.2 | 173.5 | 120.3 | 3.2 | 4.4 | 4.2 | 57.4 | 223.4 |
| Control | 0.1 | 203.0 | 171.6 | 4.4 | 7.1 | 3.2 | 81.1 | 258.4 |

The results show that a significant decrease in arachidonic acid can be found in the phospholipids in the brain (including sn-2 position) after intake of omega-3 phospholipids. DHA levels in both total lipids and phospholipids were not influenced by the omega-3 diets, while there was a small but significant increase in EPA levels. This reduction of ARA may be important as it affects the inflammatory response in the tissue, especially in pathologies were inflammation of the brain is a fundamental component such as cognitive dysfunction.

### EXAMPLE 5

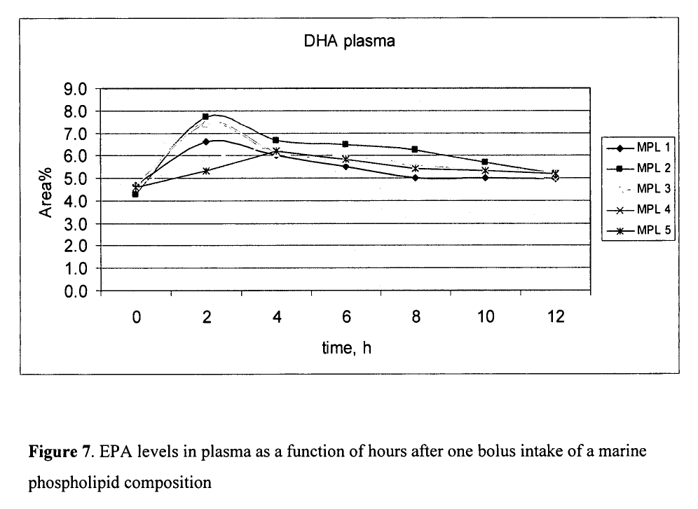

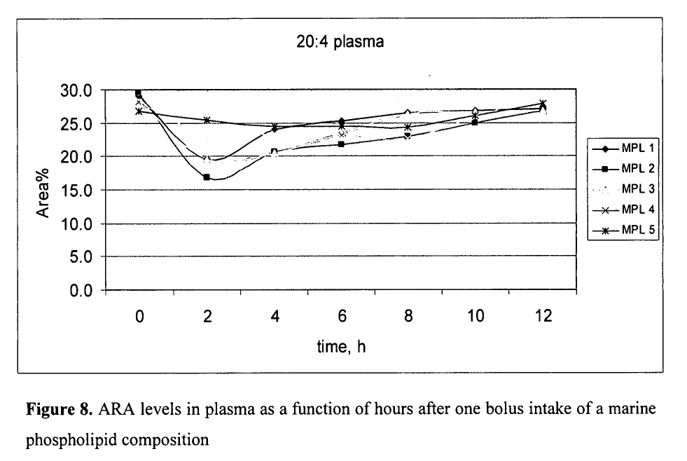

A dose-finding study aimed to investigate the effect of three dose levels (13 mg/kg, 26 mg/kg and 52 mg/kg) of the omega-3 rich phospholipids on visuospatial memory in aged beagle dogs was performed. 18 beagle dogs of age 7 years were recruited with the absence of any clinical symptoms that could affect the objectives of the study, as determined by a veterinarian. The variable measured at baseline and after 4 week were cognition as measured by the delayed non-matching-to position task (DNMP) [44]. Electroretinography (ERG) is an electrophysical technique which measures the retinal action potentials in response to light stimulation and is used to assess retinal function [49]. DNMP is a test of working memory performance and subjects will receive 10 trials daily. During the baseline phase, all subjects was given 5 cognitive test sessions on a DNMP, which provided a means of assessing visuospatial working memory and establish baseline performance levels on the spatial memory test. The baseline cognitive test performance was then used to assign animals into three cognitively equivalents groups of 6 animals per group. Each trial of the DNMP task consists of two phases. In the sample, or presentation phase, a red block is presented to the subject over one of three food wells. The subject is required to displace the block and retrieve the food in the well below the block. The block is then removed from view of the subject and a delay is initiated. At the end of the delay, the choice phase begins in which subject is presented with two identical blocks; one over the initial well and a second over one of the two remaining wells. Subjects are required to respond to the novel position to obtain the food reward. A 30-s inter-trial interval will be used to separate each trial. For the present study, all DNMP testing will consist of variable-delayed testing in which delays of 20 or 90 s will occur equally over the 10 daily test trials. The results show an improvement in cognitive function in the aged beagle dogs (FIG. 3), especially for the low (13 mg/kg) and intermediate (26 mg/kg) dose using the short delay test. For the long delay test, an improvement was observed for the low dose (13 mg/kg), whereas a reduction of cognitive performance was observed for the high dose (52 mg/kg).

The ERG's were analyzed with repeated measures ANOVA's for both response latency and response amplitude. Each analysis looked at a single variable, with baseline and treatment conditions serving as a within subject variable and dose as a between subject variable. Each of the following 10 variables were examined 1) A wave scotopic at 0 log intensity, 2) A wave scotopic at 1.2 log intensity, 3) A wave photopic at 0 log intensity, 4) A wave photopic at 1.2 log intensity, 5) B wave scotopic at −3 log intensity, 6) B wave scotopic at 1 log intensity, 7) B wave scotopic at 1.2 log intensity and 8) B wave scotopic at 30 hz frequency.

Statistically significant treatment effects in response amplitude were observed in the scotopic response in the B wave at the 0 log (p=0.02) and in the 1.2 log response (p=0.0007). The response at −3 log was marginally significant (p=0.06). These results reflect larger responses under the treatment condition than baseline. The largest effects were observed at the low and high dose. The results also revealed a significant n increase in the amplitude of the scoptopic response to the A wave at 1 and 1.2 log intensities, the treatment effect achieved statistical significance (p=0.04 and p=0.007 respectively). These changes reflected shorter onset latency in the medium dose group.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

[1] Doig S and Diks R M M. Eur J Lipid Sci Technol 105 (2003) 359.
[2] Adlercreutz P, Lyberg A M and Adlercreutz D. Eu. J Lipid. Sci Technol 105 (2003) 638.
[3] Peng L, Xu X, Mu H, Høy C E and Adler-Nissen J. Enzyme Microb Technol 31 (2002) 532.
[4] Haraldsson G G and Thorarensen A, JAOCS 75 (1999) 1143.
[5] Guo Z, Vikbjerg A F and Xu X. Biotech Adv. 23 (2005) 203.
[6] US 20040234587.
[7] U.S. Pat. No. 6,036,992.
[8] U.S. Pat. No. 5,434,183.
[9] U.S. Pat. No. 5,853,747.
[10] US 20050130937.
[11] Lemaitre-Delaunay D, Pachiaudi C, Laville M, Pousin J, Armstrong M and Lagarde M. J Lipid Res. 40 (1999) 1867.
[12] Wijendran V, Huang M, Diau G, Boehm G, Nathanielsz P W and Brenna J T. Pediatr. Res. 51 (2002) 365.
[13] Calder P. Nutr. Res. 24 (2004) 361.
[14] Wainwright, P. Br J Nutr. 83 (2000) 337.
[15] Waldron M K, Spencer A L, Bauer J E (1998) Role of long-chain polyunsaturated n-3 fatty acids in the development of the nervous system of dogs and cats. JAVMA 213(5):619-622
[16] Conquer J A, Tierney M C, Zecevic J et al. (2000). Lipids 35(12):1305-1312.
[17] Alexander J W. Nutrition 14 (1998) 627.
[18] Belluzi A, Boschi S, Brignola C, Munarini A, Cariani G and Miglio F. Am J Clin Nutr 71 (2000)339.
[19] Kremer J M. Am J Clin Nutr 71 (2000) 249.
[20] Aggarwal B B, Sethi G, Nair A and Ichikawa H. Current Signal Transduction Therapy 1 (2006) 25-52.
[21] Zhao Y, Joshi-Barve J., Barve S., Chen L. J. Am. Coll. Nutr 23 (2004) 71-78.
[22] Vargas D L, Nascimbene C, Krishnan C, Zimmerman A W, Pardo C A. Annals of Neurology 57 (2005) 67.
[23] Hauss-Wegrzyniak, B, Lukovic, L, Bigaud, M, Stoeckel, M E. Brain Research 749 (1998) 211.
[24] Licastro F and Chiappelli M. Mechanisms of Ageing and Development 124 (2003) 539.
[25] Licastro F. Expert Review of Neurotherapeutics 2 (2002) 639.
[26] Richardson A J and Montgomery P. Pediatrics 115 (2005)1360.
[27] Richardson A J. Lipids. 39 (2004) 1215.
[28] Richardson A J and Puri B K. Prog Neuropsychopharmacol Biol Psychiatry 26 (2002) 233.
[29] Burgess J R. Attention deficit hyperactivity disorder: observational and interventional studies. In: NIH workshop on omega-3 essential fatty acids and psychiatric disorders, 2-3 September 1998. Bethesda, Md.: National Institutes of Health; 1998.
[30] Peet M and Stokes C. Drugs 65 (2005) 1051.
[31] Fontani G, Corradeschi F and Felici A. Eur J Clin Invest. 35 (2005) 691.
[32] Mollica C M, Maruff P and Vance A. Hum Psychopharmacol 19 (2004) 445.
[33] WOQOL Group. The World Health Organization quality of life assessment (WHOQOL): Development and general psycometric properties. Social Science and Medicine 46 (1998) 1569.
[34] Bannach F G, Gutierrez-Fernandez A, Parmer R J and Miles L A. J Thromb Haemost. 2 (2004) 2205.
[35] Choi Y. Adv Exp Med Bio1.560 (2005) 77.
[36] Leon KG and Karsan A. Histol Histopathol. 15 (2000) 1303.
[37] Gamoh S, Hashimoto M., Hossain S, Masumura S. Clin Exp Pharmacol Physiol 28 (2001) 266.
[38] Filbum C R and Griffin D. Veterinary Therapeutics. 6 (2005) 29.
[39] Mueller R S, Fieseler K V and Fettman M J. J Small An Pract 45 (2004) 293.
[40] Heinemann K M, Waldron M K, Bigley K E and Bauer J E (2005). Improvement of Retinal Function in Canine Puppies from Mothers Fed Dietary Long Chain n-3 Polyunsaturated Fatty Acids during Gestation and Lactation (abstract) in: The American College of Veterinary Internal Medicine Meeting Conference Proceedings (ACVIM).
[41] US 20050075399
[42] US 20040028719
[43] Carrie I, Smirnova M, Clement M., De Javal D, Frances H, Bourre J M. Nutr. Neuroscience 5 (2002) 43
[44] Chan A D F, Nippak P, Murphey H., Ikeda-Douglas C, Muggenberg B, Head E, Cotman C W, Milgram N W. Behavioral Neuroscience 116 (2002) 443.
[45] Manly T., Robertson I C H, Anderson V A and Nimmo-Smith I (1999). TEA-Ch-The test of Everyday Attention for Children. Bury st Edmunds: Thames Valley Test Company.
[46] Wechsler D. (1999). The Wechsler Abbreviated Scale of Intelligence (WASI). London, UK:Harcourt Assessment.
[47] Giogia G A, Isquith P K, Goy S C, Kenworthy L. Neuropsychol. Dev. Cogn. C. Child. Neropsychol 6 (2000) 235.
[48] Conners C K, (1990). Conners' rating scales manual. New York: Multi Health Systems, Inc.
[49] Binns A and Margrain, TH. Ophthalmic Physiol Opt. 25 (2005) 492.

1. A method to reduce symptoms of cognitive dysfunction in a child comprising administering an effective amount of a marine phospholipid composition, wherein said symptoms are selected from the group consisting of ability to complete task, ability to stay on task, ability to follow instructions, ability to complete assignments, psychomotor function, long term memory, short term memory, ability to make a decision, ability to follow through on decision, ability to self-sustain attention, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information.

2. The method of claim 1, wherein said marine phospholipid composition comprises phospholipids having the following structure: 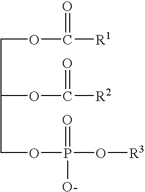
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

3. The method of claim 2, wherein said phospholipid composition comprises from about 20-50% of OH at positions R1 and/or R2.

4. The method of claim 2, wherein said phospholipid composition further comprises a lipid carrier.

5. The method of claim 1, wherein said phospholipid composition is prepared from natural marine phospholipids isolated from a marine organism.

6. The method of claim 1, wherein said phospholipid composition is enzymatically prepared by reacting lecithin with DHA and EPA in the presence of an enzyme.

7. The method of claim 6, wherein said lecithin is soybean or egg lecithin.

8. The method of claim 1, wherein said omega-3 fatty acid moieties are selected from the group of EPA and DHA and combination thereof.

9. The method of claim 1, wherein said effective amount of said phospholipid composition comprises from about 300 to about 1000 mg omega-3 fatty acids.

10. The method of claim 1, wherein said phospholipid composition is administered orally.

11. The method of claim 1, wherein said phospholipid composition is provided in a gel capsule or pill.

12. A method to reduce symptoms of cognitive dysfunction in a child with attention deficit hyperactivity disorder comprising administering an effective amount of a marine phospholipid composition, wherein said symptoms are selected from the group consisting of ability to complete task, ability to stay on task, ability to follow instructions, ability to complete assignments, psychomotor function, long term memory, short term memory, ability to make a decision, ability to follow through on decision, ability to self-sustain attention, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information.

13. The method of claim 12, wherein said marine phospholipid composition comprises phospholipids having the following structure: 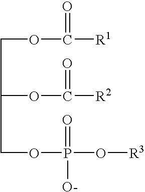
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

14. The method of claim 13, wherein said phospholipid composition comprises from about 20-50% of OH at positions R1 and/or R2.

15. The method of claim 13, wherein said phospholipid composition further comprises a lipid carrier.

16. The method of claim 12, wherein said phospholipid composition is prepared from natural marine phospholipids isolated from a marine organism.

17. The method of claim 12, wherein said phospholipid composition is enzymatically prepared by reacting lecithin with DHA and EPA in the presence of an enzyme.

18. The method of claim 17, wherein said lecithin is soybean or egg lecithin.

19. The method of claim 12, wherein said omega-3 fatty acid moieties are selected from the group of EPA and DHA and combination thereof.

20. The method of claim 12, wherein said effective amount of said phospholipid composition comprises from about 300 to about 1000 mg omega-3 fatty acids.

21. The method of claim 12, wherein said phospholipid composition is administered orally.

22. The method of claim 12, wherein said phospholipid composition is provided in a gel capsule or pill.

23. A method to reduce symptoms of cognitive dysfunction in a child with autistic spectrum disorder comprising administering an effective amount of a marine phospholipid composition, wherein said symptoms are selected from the group consisting of ability to complete task, ability to stay on task, ability to follow instructions, ability to complete assignments, psychomotor function, long term memory, short term memory, ability to make a decision, ability to follow through on decision, ability to self-sustain attention, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information.

24. The method of claim 23, wherein said marine phospholipid composition comprises phospholipids having the following structure: 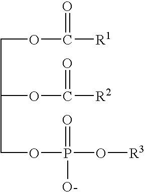
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

25. The method of claim 24, wherein said phospholipid composition comprises from about 20-50% of OH at positions R1 and/or R2.

26. The method of claim 25, wherein said phospholipid composition further comprises a lipid carrier.

27. The method of claim 23, wherein said phospholipid composition is prepared from natural marine phospholipids isolated from a marine organism.

28. The method of claim 23, wherein said phospholipid composition is enzymatically prepared by reacting lecithin with DHA and EPA in the presence of an enzyme.

29. The method of claim 28, wherein said lecithin is soybean or egg lecithin.

30. The method of claim 23, wherein said omega-3 fatty acid moieties are selected from the group of EPA and DHA and combination thereof.

31. The method of claim 23, wherein said effective amount of said phospholipid composition comprises from about 300 to about 1000 mg omega-3 fatty acids.

32. The method of claim 23, wherein said phospholipid composition is administered orally.

33. The method of claim 23, wherein said phospholipid composition is provided in a gel capsule or pill.

34. A method of increasing cognitive performance in an aging mammal comprising administering an effective amount of a marine phospholipid composition.

35. The method of claim 34, wherein said cognitive performance is selected from the group consisting of memory loss, forgetfulness, short-term memory loss, aphasia, disorientation, disinhibition, and behavioral changes.

36. The method of claim 34, wherein said mammal is a human.

37. The method of claim 34, wherein said mammal is a pet selected from the group consisting of cats and dogs.

38. The method of claim 34, wherein said mammal is has symptoms of age-associated memory impairment or decline.

39. The method of claim 34, wherein said marine phospholipid composition comprises phospholipids having the following structure: 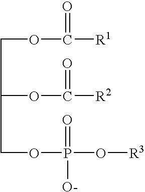
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

40. The method of claim 39, wherein said phospholipid composition comprises from about 20-50% of OH at positions R1 and/or R2.

41. The method of claim 39, wherein said phospholipid composition further comprises a lipid carrier.

42. The method of claim 34, wherein said phospholipid composition is prepared from natural marine phospholipids isolated from a marine organism.

43. The method of claim 34, wherein said phospholipid composition is enzymatically prepared by reacting lecithin with DHA and EPA in the presence of an enzyme.

44. The method of claim 43, wherein said lecithin is soybean or egg lecithin.

45. The method of claim 34, wherein said omega-3 fatty acid moieties are selected from the group of EPA and DHA and combination thereof.

46. The method of claim 34, wherein said effective amount of said phospholipid composition comprises from about 300 to about 1000 mg omega-3 fatty acids.

47. The method of claim 34, wherein said phospholipid composition is administered orally.

48. The method of claim 34, wherein said phospholipid composition is provided in a gel capsule or pill.
